(19) United States
(12) Patent Application Publication
(10) Pub. No.: US 2024/0034960 A1
(43) Pub. Date: Feb. 1, 2024

(54) ENZYMES AND ENZYME COMPOSITIONS FOR CLEANING

(71) Applicant: DANISCO US INC, PALO ALTO, CA (US)

(72) Inventors: JONATHAN LASSILA, SOUTH SAN FRANCISCO, CA (US); CLIFF LAY, MOUNTAIN VIEW, CA (US); ABIGAIL K LUCKRING, WEST CHESTER, PA (US); SERGIO ANIBAL SUNUX, SAN FRANCISCO, CA (US); WEI WEI, SHANGHAI (CN); SANDY ZHONG, SHANGHAI (CN)

(21) Appl. No.: 18/042,307

(22) PCT Filed: Aug. 27, 2021
(86) PCT No.: PCT/US2021/047937

§ 371 Date: Feb. 21, 2023

(30) Foreign Application Priority Data
Aug. 27, 2020 (WO) PCT/CN2020/111677

(51) Int. Cl. C11D 3/386; C12N 9/36; C11D 11/00

(52) U.S. Cl. CPC C11D 3/38636; C11D 3/38663; C12N 9/2462; C12Y 302/01017; C11D 11/0017; C11D 11/0023

(57) ABSTRACT

Disclosed herein are compositions and methods for preventing, reducing, or removing biofilms and microbial growth from liquid solutions, as well as from surfaces, such as fabrics, textiles or hard surfaces.

### CROSS REFERENCE

This application is a 371 of international application No. PCT/US2021/047937, filed Aug. 27, 2021 and claims priority benefit of PCT/CN2020/111677, filed Aug. 27, 2020, each of which is incorporated by reference in its entirety.

The present disclosure relates to compositions and methods for cleaning, for example hard surface and laundry cleaning.

### REFERENCE TO SEQUENCE LISTING SUBMITTED ELECTRONICALLY

The official copy of the sequence listing is submitted electronically via the Patent Center as an XML, formatted sequence listing with a file named 20230221_NB41608USPCT2_SeqLst created on Feb. 8, 2023 and having a size of 28399 bytes and is filed concurrently with the specification. The sequence listing contained in this XML formatted document is part of the specification and is herein incorporated by reference in its entirety.

### BACKGROUND

Trends toward cold water washing and synthetic athletic wear are driving a need for detergents that eliminate bacteria and odor, while at the same time the industry is moving away from laundry powders where traditional oxygen bleach was feasible. Despite exposure to surfactants, proteases, amylases, and mechanical washing from typical laundry processes, odor and microorganisms persist on clothes and washing machines. New solutions are thus needed to remove odors and microorganisms in the laundry.

The formation of biofilms further complicates the challenge of removing odors and microorganisms, as odor compounds and bacteria may become resistant to removal by traditional laundry detergents when embedded in extracellular matrix from biofilms. Traditional odor capture agents or antimicrobials may not be able to act in the presence of these built-up films on textiles and washing machines. Thus, new solutions are also needed for odor and microorganism removal in the presence of this type of persistent soiling caused by microorganisms.

Beyond laundry, new solutions to cleaning, malodor reduction, and microbial load reduction are needed for many different applications, including in personal care, in food and beverage preparation and packaging, in industrial settings, and in medical and oral care.

### SUMMARY

One embodiment is directed to an isolated polypeptide or active fragment thereof having lysozyme activity, where the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 11, 12, 13, 14, 15, 16, 17, 18, 19, 20, 21, 22, 23, 24, and 25.

In another embodiment, the present disclosure provides an isolated polynucleotide comprising a nucleotide sequence that encodes a polypeptide having lysozyme activity, where the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 21, 22, 23, 24, and 25.

A further embodiment of the present disclosure provides a recombinant nucleic acid construct comprising an isolated polynucleotide comprising a nucleotide sequence that encodes a polypeptide having lysozyme activity, where the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25 operably linked to a promoter sequence capable of controlling expression of the polynucleotide sequence.

In yet another embodiment, the present disclosure provides an isolated host cell comprising a recombinant nucleic acid construct comprising an isolated polynucleotide comprising a nucleotide sequence that encodes a polypeptide having lysozyme activity, where the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 21, 22, 23, 24, and 25 operably linked to a promoter sequence capable of controlling expression of the polynucleotide sequence.

The present disclosure also provides methods for producing a polypeptide having lysozyme activity, the method comprising: a) cultivating a host cell comprising a recombinant nucleic acid construct comprising an isolated polynucleotide comprising a nucleotide sequence that encodes a polypeptide having lysozyme activity, where the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25 operably linked to a promoter sequence capable of controlling expression of the polynucleotide sequence under conditions conducive to producing the polypeptide, and b) optionally, recovering the polypeptide having lysozyme activity.

Also provided herein are methods for preventing, reducing or removing a biofilm comprising contacting the biofilm with a polypeptide having lysozyme activity or a composition comprising a polypeptide having lysozyme activity. In some such embodiments, the polypeptide having lysozyme activity has an amino acid sequence having at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

In another embodiment, the present disclosure provides methods for preventing, reducing or removing a biofilm and/or preventing, reducing or removing microbial growth on a textile or hard surface comprising: (i) contacting a textile or surface with a polypeptide having lysozyme activity or a composition comprising a polypeptide having lysozyme activity; and (ii) optionally, rinsing the textile or surface. In some such embodiments, the polypeptide having lysozyme activity has an amino acid sequence having at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 11, 12, 13, 14, 15, 16, 17, 18, 19, 20, 21, 22, 23, 24, and 25.

The present disclosure further provides detergent compositions comprising: (i) a polypeptide having lysozyme activity; (ii) a polypeptide having protease activity; (iii) optionally, at least one additional polypeptide, where the at least one additional polypeptide is an enzyme selected from: acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof; and (iv) a surfactant.

Also provided are methods for preventing, reducing or removing microbial growth in a liquid detergent solution comprising including in a liquid detergent solution an effective amount of a lysozyme and a surfactant. In some such embodiments, the lysozyme has an amino acid sequence having at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

Also provided are compositions comprising at least 0.002 mg of a polypeptide having lysozyme activity, where the polypeptide has an amino acid sequence having at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

The present disclosure also provides methods for preventing, reducing or removing microbial growth in a liquid composition comprising including in the composition an effective amount of a lysozyme, where the lysozyme has an amino acid sequence having at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

The present disclosure further provides for the use of a lysozyme for preventing, reducing, or removing microbial growth in a liquid detergent.

### FIGURES

FIG. 1 provides a graphic representation of the results of one embodiment of the present disclosure providing data demonstrating a reduction of Pseudomonas fluorescens bacterial biofilm by T4 lysozyme. Black circle indicates simulated wash solution with no lysozyme. Light gray circle indicates untreated control. Dark gray circles indicate treatment with the given concentration of T4 lysozyme in the simulated wash solution.

FIG. 2 provides a graphic representation of the results of one embodiment of the present disclosure providing data demonstrating the reduction of Pseudomonas fluorescens biofilms following treatment with lysozymes for 2 hours. Error bars indicate standard deviations from eight replicates.

FIG. 3 provides a graphic representation of the results of one embodiment of the present disclosure providing data demonstrating the reduction of Pseudomonas fluorescens biofilms following treatment with lysozymes in detergent wash solution for 400 minutes. Black bars indicate either 10 PPM enzyme or no-enzyme controls, as indicated. Medium gray bars indicate 50 PPM enzyme. Light gray bars indicate 250 PPM enzyme. Error bars indicate standard deviations from eight replicates.

FIG. 4 provides a graphic representation of the results of one embodiment of the present disclosure providing the results of liquid culture outgrowth tests showing reduction of Micrococcus luteus bacteria following exposure to varying concentrations of lysozymes.

FIG. 5 provides a graphic representation of the results of one embodiment of the present disclosure providing the results of liquid culture outgrowth tests showing reduction of Moraxella osloensis bacteria following exposure to varying concentrations of lysozymes.

FIG. 6 provides a graphic representation of the results of one embodiment of the present disclosure providing data demonstrating the reduction of Pseudomonas fluorescens biofilms following treatment with lysozymes in detergent wash solution for 400 minutes.

FIG. 7 provides a graphic representation of the results of one embodiment of the present disclosure providing data demonstrating the reduction of Micrococcus lysodeikticus cells following exposure to lysozymes, relative to a no-enzyme control.

### DESCRIPTION

The present disclosure provides polypeptides having lysozyme activity, compositions (e.g. enzyme and detergent compositions) comprising such polypeptides, and methods using such compositions for the prevention, reduction or removal of microorganisms or biofilms, for example, from an article, such as a hard surface or textile. The compositions generally employ the use of at least one polypeptide having lysozyme activity or an active fragment thereof or a composition comprising a polypeptide having lysozyme activity or active fragment thereof. The compositions also optionally comprise additional components of a cleaning detergent, such as one or more surfactants.

Prior to describing embodiments of present compositions and methods, the following terms are defined.

Unless defined otherwise herein, all technical and scientific terms used herein have the same meaning as commonly understood by one of ordinary skill in the art to which this invention pertains. Although any methods and materials similar or equivalent to those described herein find use in the practice of the present invention, the preferred methods and materials are described herein. Accordingly, the terms defined immediately below are more fully described by reference to the specification as a whole. Also, as used herein, the singular terms “a,” “an,” and “the” include the plural reference unless the context clearly indicates otherwise. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The term “biofilm” refers to a community of microorganisms embedded in an extracellular polymer matrix attached to a surface. The extracellular polymer matrix is a polymeric conglomeration generally composed of extracellular DNA, proteins, and polysaccharides. A biofilm may have one or more microorganisms and further includes water and may include other trapped particles. The microorganisms may be gram positive or gram-negative bacteria (aerobic or anaerobic); algae, protozoa, and/or yeast or filamentous fungi and combinations thereof. In some embodiments the biofilm may include living cells including one or more bacterial genera of Acinetobacter sp., Aeromicrobium sp., Brevundimonas sp., Burkholderia sp., Campylobacter sp., Clostridium sp., Desulfovibrio sp., Escherichia sp., Raemophilus sp., Lactobacillus sp., Laciococcus sp., Listeria sp., Microbacterium sp., Micrococcus sp., (e.g. Alicrococcus luteus), Moraxella sp., (e.g. Moraxella osloensis), Porphyromonas sp., Priopionibacterium sp., Pseudomonas sp. (e.g. Pseudomonas fluorescens, Pseudomonas putida, Pseudomonas aeruginosa), Salmonella sp. Staphylococcus sp. (e.g. Staphylococcus epidermidis, Staphylococcus aureus), and Stenotrophomonas sp., Streptomyces sp., Listeria sp., Streptococcus sp. (e.g. Streptococcus mutans), and Vibrio, sp., or yeast such as Candida sp.

As used herein, “surface” means any structure having sufficient mass to allow for attachment of biofilm, including hard surfaces, soft surfaces, porous surfaces and other types of surfaces. Hard surfaces include, but are not limited to metal, glass, ceramics, wood, minerals (rock, stone, marble, granite), aggregate materials such as concrete, plastics, composite materials, hard rubber materials, and gypsum. The hard materials may be finished with enamels and paints. Hard surfaces are found, for example in water treatment and storage equipment and tanks; dairy and food processing equipment and facilities; medical equipment and facilities, such as surgical instruments and permanent and temporary implants; industrial pharmaceutical equipment and plants. Soft surfaces are, for example, hair and all types of textiles. Porous surfaces may be biological surfaces, such as skin, keratin or internal organs. Porous surfaces also may be found in certain ceramics as well as in membranes that are used for filtration. Other surfaces include, but are not limited to, ship hulls and swimming pools.

The term “fabric” refers to, for example, woven, knit, and non-woven material, as well as staple fibers and filaments that can be converted to, for example, yarns and woven, knit, and non-woven fabrics. The term encompasses material made from natural, as well as synthetic (e.g., manufactured) fibers.

The term “textile”, as used herein, refers to any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastase, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polvurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used, it is intended to include the broader term textiles as well. In the context of the present application, the term “textile” is used interchangeably with fabric and cloth.

As used herein, the term “hard surface” refers to any article having a hard surface including floors, tables, walls, roofs etc. as well as surfaces of hard objects such as cars (car wash), ship hulls, dishes (dishware), medical instruments, pipes, reservoirs, or holding tanks. The term “hard surface” includes also the surfaces of flexible yet firm objects such as the insides of bendable tubing and supply lines or the surfaces of deformable holding tanks or vessels. The term “hard surface” includes also the surfaces in the interior of washing machines, such as the interior of laundry washing machines or dishwashing machines, this includes soap intake box, walls, windows, baskets, racks, nozzles, pumps, sump, filters, pipelines, tubes, joints, seals, gaskets, fittings, impellers, drums, drains, traps, coin traps inlet and outlets. The term hard surface does not encompass textile or fabric.

The term “laundering” includes both household laundering and industrial laundering and means the process of treating textiles with a solution containing a cleaning or detergent composition as provided herein. The laundering process can for example be carried out using e.g. a household or an industrial washing machine or can be carried out by hand.

The term “wash cycle” refers to a washing operation in which textiles are immersed in a wash liquor, mechanical action of some kind is applied to the textile to release stains or to facilitate flow of wash liquor in and out of the textile and finally the superfluous wash liquor is removed. After one or more wash cycles, the textile is generally rinsed and dried.

The term “wash liquor” is defined herein as the solution or mixture of water and detergent components optionally including polypeptides having lysozyme activity.

### Polypeptides

In one embodiment, polypeptides are provided that have lysozyme activity. The polypeptides having lysozyme activity of the present disclosure include isolated, recombinant, substantially pure, or non-naturally occurring polypeptides. In some embodiments, the polypeptides are useful in cleaning applications and can be incorporated into cleaning compositions that are useful in methods of cleaning an item or a surface in need thereof.

The lysozyme polypeptide for use in the methods and compositions herein includes any lysozyme polypeptide. As used herein, the term “lysozyme” refers to any polypeptide or fragment thereof that is capable of hydrolyzing N-acetylmuramoyl-β-1,4-N-acetylglucosamine bonds to degrade bacterial peptidoglycan (Schmelcher et al 2012, Future Microbiol 7:1147-1171; Loessner 2005, Current Opinion in Microbiology 8: 480-487; Thallinger et al 2013, Biotechnol J 9:97-109). In some embodiments, the polypeptide having lysozyme activity for use in the compositions and methods provided herein include those having amino acid sequences having at least 50%, 55%, 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

As used herein, the term “variant polypeptide” refers to a polypeptide comprising an amino acid sequence that differs in at least one amino acid residue from the amino acid sequence of a parent or reference polypeptide (including but not limited to wild-type polypeptides)

In some embodiments, the lysozyme polypeptides provided herein have an amino acid sequence at least 50%, 55%, 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25. In some embodiments, the lysozyme polypeptides provided herein have an amino acid sequence at least 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 6, 7, 8, 9, 10, 11, 14, 19, 22, 23, and 25. In some embodiments, the lysozyme polypeptides provided herein have an amino acid sequence at least 90%, 91%, 92%, 93%, 94%, 95%, 96%, 97%, 98%, 99%, or 100% sequence identity with the amino acid sequence selected from the group consisting of SEQ ID NOs: 15, 16, 20, and 21. In some embodiments, the lysozyme polypeptides provided herein have an amino acid sequence at least 95%, 96%, 97%, 98%, 99%, or 100% sequence identity with the amino acid sequence selected from the group consisting of SEQ ID NOs: 12.

In some embodiments, the lysozyme for use in the compositions and methods provided herein includes a polypeptide having an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25. In some embodiments, the lysozyme has an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25, and has lysozyme activity.

As used herein, “% identity or percent identity” refers to sequence similarity. Percent identity may be determined using standard techniques known in the art (See e.g., Smith and Waterman, Adv. Appl. Math. 2:482 [1981]; Needleman and Wunsch, J. Mol. Biol. 48:443 [1970]; Pearson and Lipman, Proc. Natl. Acad. Sci. USA 85:2444 [1988]; software programs such as GAP, BESTFIT, FASTA, and TFASTA in the Wisconsin Genetics Software Package (Genetics Computer Group, Madison, WI); and Devereux et al., Nucl. Acid Res. 12:387-395 [1984]). One example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (See, Feng and Doolittle, J. Mol. Evol. 35:351-360 [1987]). The method is similar to that described by Higgins and Sharp (See, Higgins and Sharp, CABIOS 5:151-153 [1989]). Useful PILEUP parameters include a default gap weight of 3.00, a default gap length weight of 0.10, and weighted end gaps. Other useful algorithm is the BLAST algorithms described by Altschul et al., (See, Altschul et al., J. Mol. Biol. 215:403-410 [1990]; and Karlin and Altschul, Proc. Natl. Acad. Sci. USA 90:5873-5787 [1993]). The BLAST program uses several search parameters, most of which are set to the default values.

As used herein, “homologous proteins” or “homologous lysozymes” refers to proteins that have distinct similarity in primary, secondary, and/or tertiary structure. Protein homology can refer to the similarity in linear amino acid sequence when proteins are aligned. Homology can be determined by amino acid sequence alignment, e.g., using a program such as BLAST, MUSCLE, or CLUSTAL. Homologous search of protein sequences can be done using BLASTP and PSI-BLAST from NCBI BLAST with threshold (E-value cut-off) at 0.001. (Altschul et al., “Gapped BLAST and PSI BLAST a new generation of protein database search programs”, Nucleic Acids Res, Set 1;25(17):3389-402(1997)). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997 and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=-3); Gap Opening=5; and Gap Extension=2. Exemplary default BLAST parameters for amino acid sequence searches include: Word size =3; E-value cutoff=10; Scoring Matrix=BLOSUM62; Gap Opening=11; and Gap extension=1. Using this information, protein sequences can be grouped and/or a phylogenetic tree built therefrom. Amino acid sequences can be entered in a program such as the Vector NTI Advance suite and a Guide Tree can be created using the Neighbor Joining (NJ) method (Saitou and Nei, Mol Biol Evol, 4:406-425, 1987). The tree construction can be calculated using Kimura's correction for sequence distance and ignoring positions with gaps. A program such as AlignX can display the calculated distance values in parentheses following the molecule name displayed on the phylogenetic tree.

A percent (%) amino acid sequence identity value is determined by the number of matching identical residues divided by the total number of residues of the “reference” sequence including any gaps created by the program for optimal/maximum alignment. If a sequence is 90% identical to SEQ ID NO: A, SEQ ID NO: A is the “reference” sequence. BLAST algorithms refer the “reference” sequence as “query” sequence.

The CLUSTAL W algorithm is another example of a sequence alignment algorithm (See, Thompson et al., Nucleic Acids Res, 22:4673-4680, 1994). Default parameters for the CLUSTAL W algorithm include: Gap opening penalty=10.0; Gap extension penalty=0.05; Protein weight matrix=BLOSUM series; DNA weight matrix=IUB; Delay divergent sequences %=40; Gap separation distance=8; DNA transitions weight=0.50; List hydrophilic residues=GPSNDQEKR; Use negative matrix=OFF; Toggle Residue specific penalties=ON; Toggle hydrophilic penalties=ON; and Toggle end gap separation penalty=OFF. In CLUSTAL algorithms, deletions occurring at either terminus are included. For example, a variant with a five amino acid deletion at either terminus (or within the polypeptide) of a polypeptide of 500 amino acids would have a percent sequence identity of 99% (495/500 identical residues x 100) relative to the “reference” polypeptide. Such a variant would be encompassed by a variant having “at least 99% sequence identity” to the polypeptide.

In some embodiments, the polypeptide of the present invention is a polypeptide having a specified degree of amino acid sequence homology to the exemplified polypeptides, e.g., 50%, 55%, 60%, 65%, 70%, 75%, 80%, 85%, 90%, 91%, 92%, 93%, 94%, 95%, 96%, 97%, 98%, 99% or 100% amino acid sequence identity to an amino acid sequence selected from the group consisting of SEQ ID NOs: 1, 2, 3, 4, 5, 6, 7, 8, 9, 10, 11, 12, 13, 14, 15, 16, 17, 18, 19, 21, 22, 23, 24, and 25. In some embodiments, the recombinant polypeptide or active fragment thereof comprises an amino acid sequence having at least 70% amino acid sequence identity to the amino acid sequence of SEQ ID NOs: 1, 2, 3, 4, 5, 6, 7, 8, 9, 10, 11, 12, 13, 14, 16, 17, 18, 19, 20, 21, 22, 23, 24, and 25. In some embodiments, the recombinant polypeptide or active fragment thereof comprises an amino acid sequence having at least 75% amino acid sequence identity to the amino acid sequence of SEQ ID NOs: 1, 2, 3, 4, 5, 6, 7, 8, 9, 10, 11, 12, 13, 14, 15, 16, 17, 18, 19, 20, 21, 22, 23, 24, and 25. In some embodiments, the recombinant polypeptide or active fragment thereof comprises an amino acid sequence having at least 80%, 90%, or 95% amino acid sequence identity to the amino acid sequence of SEQ ID NOs: 1, 2, 3, 4, 5, 6, 7, 8, 9, 10, 11, 12, 13, 14, 15, 16, 17, 18, 19, 20, 21, 22, 23, 24, and 25. Homology can be determined by amino acid sequence alignment, e.g., using a program such as BLAST, ALIGN, or CLUSTAL, as described herein. In some embodiments, the polypeptide is an isolated, recombinant, substantially pure, or non-naturally occurring enzyme having lysozyme activity, such as N-acetylmuramoyl-β-1,4-N-acetylglucosamine hydrolysis activity and/or bacterial peptidoglycan degradation activity.

Also provided is a variant lysozyme polypeptide enzyme, having lysozyme activity, where the enzyme comprises an amino acid sequence which differs from the amino acid sequence of SEQ ID NOs: 1, 2, 3, 4, 5, 6, 7, 8, 9, 10, 11, 12, 13, 14, 15, 16, 17, 18, 19, 20, 21, 22, 23, 24, and 25 by no more than 50, no more than 40, no more than 30, no more than 25, no more than 20, no more than 15, no more than 10, no more than 9, no more than 8, no more than 7, no more than 6, no more than 5, no more than 4, no more than 3, no more than 2, or no more than 1 amino acid residue(s), when aligned using any of the previously described alignment methods.

The variant enzyme polypeptides of the disclosure have enzymatic activities (e.g., lysozyme activities) and thus are useful in a variety of cleaning applications, including but not limited to, methods for cleaning dishware items, tableware items, fabrics, textiles, and items having hard surfaces (e.g., the hard surface of a table, table top, wall, furniture item, floor, ceiling, etc.). Exemplary cleaning compositions comprising one or more polypeptides having lysozyme activity of the disclosure are described infra. The enzymatic activity (e.g., lysozyme activity) of an enzyme polypeptide of the invention can be determined readily using procedures well known to those of ordinary skill in the art. The Examples presented infra describe methods for evaluating the enzymatic activity and cleaning performance. The performance of polypeptide enzymes of the invention in reducing, preventing, and/or removing a biofilm can be readily determined using procedures well known in the art and/or by using procedures set forth in the Examples.

In some embodiments, the polypeptides of the present disclosure can have lysozyme activity over a broad range of pH conditions. In some embodiments, the polypeptides have lysozyme activity as demonstrated using the methods described in the examples. In some embodiments, the polypeptides have lysozyme activity as demonstrated using activity assays available commercially or described in the literature, such as the EnzChek® Lysozyme Assay Kit (ThermoFisher) or that described by Gorin et al (Gorin, G., Wang, S. F., Papapavlou, L (1971) Assay of lysozyme by its lytic action on M-lysodeikticus cells. Analytical Biochemistry 39:113-127). In some embodiments, the polypeptides have lysozyme activity at a pH of from about 4.0 to about 12.0. In some embodiments, the polypeptides have lysozyme activity at a pH of from about 6.0 to about 12.0. In some embodiments, the polypeptides have at least 50%, 60%, 70%, 80% or 90% of maximal lysozyme activity at a pH of from about 6.0 to about 12.0, or from about 7.0 to about 12.0, or at a pH of from about 6 to about 10, or at a pH of from about 6 to about 9. In some embodiments, the polypeptides have lysozyme activity at a pH above 6.0, 6.5, 7.0, 7.5, 8.0, 8.5, 9.0, 9.5, 10.0, 10.5, 11.0 or 11.5. In some embodiments, the polypeptides have lysozyme activity at a pH below 12.0, 11.5, 11.0, 10.5, 10.0, 9.5, 9.0, 8.5, 8.0, 7.5, 7.0, or 6.5.

In some embodiments, the polypeptides of the present disclosure have lysozyme activity at a temperature range from about 10° C. to about 90° C., or from about 20° C. to about 40° C. In some embodiments, the polypeptides of the present disclosure have lysozyme activity at a temperature range of from about 20° C. to about 40° C. In some embodiments, the polypeptides have at least 50%, 60%, 70%, 80% or 90% of maximal lysozyme activity at a temperature of from about 20° C. to about 40° C. In some embodiments, the polypeptides have activity at a temperature above 50° C., 55° C., 60° C., 65° C., or 70° C. In some embodiments, the polypeptides have activity at a temperature below 90° C., 85° C., 80° C., 75° C., 70° C., 65° C., 60° C., or 55° C.

A lysozyme polypeptide of the present disclosure can be subject to various changes, such as one or more amino acid insertions, deletions, and/or substitutions, either conservative or non-conservative, including where such changes do not substantially alter the enzymatic activity of the polypeptide. Similarly, a nucleic acid of the invention can also be subject to various changes, such as one or more substitutions of one or more nucleotides in one or more codons such that a particular codon encodes the same or a different amino acid, resulting in either a silent variation (e.g., when the encoded amino acid is not altered by the nucleotide mutation) or non-silent variation, one or more deletions of one or more nucleic acids (or codons) in the sequence, one or more additions or insertions of one or more nucleic acids (or codons) in the sequence, and/or cleavage of or one or more truncations of one or more nucleic acids (or codons) in the sequence. Many such changes in the nucleic acid sequence may not substantially alter the enzymatic activity of the resulting encoded polypeptide enzyme compared to the polypeptide enzyme encoded by the original nucleic acid sequence. A nucleic acid sequence of the invention can also be modified to include one or more codons that provide for optimum expression in an expression system (e.g., bacterial expression system), while, if desired, said one or more codons still encode the same amino acid(s).

The disclosure provides isolated, non-naturally occurring, or recombinant nucleic acids which may be collectively referred to as “nucleic acids” or “polynucleotides”, which encode polypeptides of the disclosure. Nucleic acids of the disclosure, including all described below, are useful in recombinant production (e.g., expression) of polypeptides of the disclosure, typically through expression of a plasmid expression vector comprising a sequence encoding the polypeptide of interest or fragment thereof. As discussed above, polypeptides of the present disclosure include polypeptides having enzymatic activity (e.g., lysozyme activity) which are useful in cleaning applications and cleaning compositions for cleaning an item or a surface (e.g., surface of an item) in need of cleaning and/or reducing, removing or preventing biofilms, or for removing microorganisms from an item, a surface, or a solution.

In some embodiments, the polynucleotide of the present disclosure is a polynucleotide having a specified degree of nucleic acid homology to the exemplified polynucleotide. In some embodiments, the polynucleotide has a nucleic acid sequence that encodes a polypeptide or an active fragment thereof having at least 70, 75, 80, 85, 90, 91, 92, 93, 94, 95, 96, 97, 98, 99, or 100% amino acid sequence identity to an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25. Homology can be determined by amino acid sequence alignment, e.g., using a program such as BLAST, ALIGN, or CLUSTAL, as described herein.

In some embodiments, the disclosure provides an isolated, recombinant, substantially pure, synthetically derived, or non-naturally occurring nucleic acid comprising a nucleotide sequence encoding any polypeptide (including any fusion protein, etc.) having lysozyme activity described herein. The disclosure also provides an isolated, recombinant, substantially pure, synthetically derived, or non-naturally-occurring nucleic acid comprising a nucleotide sequence encoding a combination of two or more of any polypeptides provided herein. The present disclosure provides nucleic acids encoding a polypeptide having lysozyme activity of the present disclosure, wherein the polypeptide is a mature form having lysozyme activity. In some embodiments, the polypeptide is expressed recombinantly with a homologous pro-peptide sequence. In other embodiments, the polypeptide is expressed recombinantly with a heterologous pro-peptide sequence.

The nucleic acids provided herein can be generated by using any suitable synthesis, manipulation, and/or isolation techniques, or combinations thereof. For example, a polynucleotide provided herein may be produced using standard nucleic acid synthesis techniques, such as solid-phase synthesis techniques that are well-known to those skilled in the art. In such techniques, fragments of up to 50 or more nucleotide bases are typically synthesized, then joined (e.g., by enzymatic or chemical ligation methods) to form essentially any desired continuous nucleic acid sequence. The synthesis of the nucleic acids can be also facilitated by any suitable method known in the art, including but not limited to chemical synthesis using the classical phosphoramidite method (See e.g., Beaucage et al. Tetrahedron Letters 22:1859-69 [1981]; or the method described by Matthes et al. (See, Matthes et al., EMBO J. 3:801-805 [1984], as is typically practiced in automated synthetic methods. Nucleic acids of the invention also can be produced by using an automatic DNA synthesizer. Customized nucleic acids can be ordered from a variety of commercial sources (e.g., The Midland Certified Reagent Company, the Great American Gene Company, Operon Technologies Inc., and DNA2.0). Other techniques for synthesizing nucleic acids and related principles are known in the art (See e.g., Itakura et al., Ann. Rev. Biochem. 53:323 [1984]; and Itakura et al., Science 198:1056 [1984]).

The present disclosure also provides recombinant vectors comprising at least one polynucleotide described herein (e.g., a polynucleotide encoding a polypeptide having lysozyme activity provided herein), expression vectors or expression cassettes comprising at least one nucleic acid or polynucleotide of the disclosure, isolated, substantially pure, or recombinant DNA constructs comprising at least one nucleic acid or polynucleotide of the disclosure, isolated or recombinant cells comprising at least one polynucleotide of the disclosure, and compositions comprising one or more such vectors, nucleic acids, expression vectors, expression cassettes, DNA constructs, cells, cell cultures, or any combination or mixtures thereof. In some embodiments, the disclosure provides recombinant cells comprising at least one vector (e.g., expression vector or DNA construct) which comprises at least one nucleic acid or polynucleotide provided herein. Some such recombinant cells are transformed or transfected with such at least one vector, although other methods are available and known in the art. Such cells are typically referred to as host cells. Some such cells comprise bacterial cells, including, but are not limited to Bacillus sp. cells, such as B. subtilis cells. Some such cells comprise fungal cells, including but not limited to Trichoderma cells, such as Trichoderma reesei cells. The disclosure also provides recombinant cells (e.g., recombinant host cells) comprising at least one polypeptide having lysozyme activity of the disclosure.

In some embodiments, the disclosure provides a vector comprising a nucleic acid or polynucleotide as described herein. In some embodiments, the vector is an expression vector or expression cassette in which a polynucleotide sequence which encodes a polypeptide having lysozyme activity is operably linked to one or additional nucleic acid segments required for efficient gene expression (e.g., a promoter operably linked to the polynucleotide of the invention which encodes a serine protease polypeptide of the invention). A vector may include a transcription terminator and/or a selection gene, such as an antibiotic resistance gene, that enables continuous cultural maintenance of plasmid-infected host cells by growth in antimicrobial-containing media.

An expression vector may be derived from plasmid or viral DNA, or in alternative embodiments, contains elements of both. Exemplary vectors include, but are not limited to pC194, pJH101, pE194, pHP13 (See, Harwood and Cutting [eds.], Chapter 3, Molecular

Biological Methods for Bacillus, John Wiley & Sons [1990]; suitable replicating plasmids for B. subtilis include those listed on p. 92) See also, Perego, Integrational Vectors for Genetic Manipulations in Bacillus subtilis, in Sonenshein et al., [eds.] Bacillus subtilis and Other Gram-Positive Bacteria: Biochemistry, Physiology and Molecular Genetics, American Society for Microbiology, Washington, D.C. [1993], pp. 615-624), and p2JM103BBI.

For expression and production of a protein of interest (e.g., a polypeptide having lysozyme activity) in a cell, at least one expression vector comprising at least one copy of a polynucleotide encoding the polypeptide having lysozyme activity, and in some instances comprising multiple copies, is transformed into the cell under conditions suitable for expression of the polypeptide. In some embodiments, a polynucleotide sequence encoding the polypeptide having lysozyme activity (as well as other sequences included in the vector) is integrated into the genome of the host cell, while in other embodiments, a plasmid vector comprising a polynucleotide sequence encoding the polypeptide having lysozyme activity remains as autonomous extra-chromosomal element within the cell. The disclosure provides both extrachromosomal nucleic acid elements as well as incoming nucleotide sequences that are integrated into the host cell genome. The vectors described herein are useful for production of the polypeptides having lysozyme activity as provided herein. In some embodiments, a polynucleotide construct encoding the polypeptide is present on an integrating vector that enables the integration and optionally the amplification of the polynucleotide encoding the polypeptide into the host chromosome. Examples of sites for integration are well known to those skilled in the art. In some embodiments, transcription of a polynucleotide encoding a polypeptide of the disclosure is effectuated by a promoter that is the wild-type promoter for the selected precursor lysozyme. In some other embodiments, the promoter is heterologous to the precursor lysozyme, but is functional in the host cell. Specifically, examples of suitable promoters for use in bacterial host cells include, but are not limited to, for example, the amyE, amyQ, amyL, pstS, sacB, pSPAC, pAprE, pVeg, pHpall promoters, the promoter of the B. stearothermophilus maltogenic amylase gene, the B. amyloliquefaciens (BAN) amylase gene, the B. subtilis alkaline protease gene, the B. clausii alkaline protease gene the B. pumilis xylosidase gene, the B. thuringiensis cryIIIA, and the B. licheniformis alpha-amylase gene. Additional promoters include, but are not limited to the A4 promoter, as well as phage Lambda PR or PL promoters, and the E. coli lac, trp or tac promoters.

The polypeptides of the present disclosure can be produced in host cells of any suitable microorganism, including bacteria and fungi. In some embodiments, the polypeptides of the present disclosure can be produced in Gram-positive bacteria. In some embodiments, the host cells are Bacillus spp., Streptomyces spp., Escherichia spp., Aspergillus spp., Trichoderma spp., Pseudomonas spp., Corynebacterium spp., Saccharomyces spp., or Pichia spp. In some embodiments, the polypeptides are produced by Bacillus sp. host cells. Examples of Bacillus sp. host cells that find use in the production of the polypeptides of the invention include, but are not limited to B. licheniformis, B. lentus, B. subtilis, B. amyloliquefaciens, B. lentus, B. brevis, B. stearothermophilus, B. alkalophilus, B. coagulans, B. circulans, B. pumilis, B. thuringiensis, B. clausii, and B. megaterium, as well as other organisms within the genus Bacillus. In some embodiments, B. subtilis host cells are used for production of the polypeptides having lysozyme activity. U.S. Pat. Nos. 5,264,366 and 4,760,025 (RE 34,606) describe various Bacillus host strains that can be used for producing the polypeptide of the disclosure, although other suitable strains can be used.

Several bacterial strains that can be used to produce polypeptides of the disclosure include non-recombinant (i.e., wild-type) Bacillus sp. strains, as well as variants of naturally-occurring strains and/or recombinant strains. In some embodiments, the host strain is a recombinant strain, wherein a polynucleotide encoding a polypeptide of interest has been introduced into the host. In some embodiments, the host strain is a B. subtilis host strain and particularly a recombinant B. subtilis host strain. Numerous B. subtilis strains are known, including, but not limited to for example, 1A6 (ATCC 39085), 168 (1A01), SB19, W23, Ts85, B637, PB1753 through PB1758, PB3360, JH642, 1A243 (ATCC 39,087), ATCC 21332, ATCC 6051, MI113, DE100 (ATCC 39,094), GX4931, PBT 110, and PEP 211strain (See e.g., Hoch et al., Genetics 73:215-228 [1973]; See also, U.S. Pat. Nos. 4,450,235 and 4,302,544, and EP 0134048, each of which is incorporated by reference in its entirety). The use of B. subtilis as an expression host cell is well known in the art (See e.g., Palva et al., Gene 19:81-87 [1982]; Fahnestock and Fischer, J. Bacteriol., 165:796-804 [1986]; and Wang et al., Gene 69:39-47 [1998]).

In some embodiments, the Bacillus host cell is a Bacillus sp. that includes a mutation or deletion in at least one of the following genes, degU, degS, degR and degQ. In some embodiments, the mutation is in a degU gene, and in some embodiments the mutation is degU(Hy)32 (See e.g., Msadek et al., J. Bacteriol. 172:824-834 [1990]; and Olmos et al., Mol. Gen. Genet. 253:562-567 [1997]). In some embodiments, the Bacillus host comprises a mutation or deletion in scoC4 (See e.g., Caldwell et al., J. Bacteriol. 183:7329-7340 [2001]); spoIIE (See e.g., Arigoni et al., Mol. Microbiol. 31:1407-1415 [1999]); and/or oppA or other genes of the opp operon (See e.g., Perego et al., Mol. Microbiol. 5:173-185 [1991]). Indeed, it is contemplated that any mutation in the opp operon that causes the same phenotype as a mutation in the oppA gene will find use in some embodiments of the altered Bacillus strain. In some embodiments, these mutations occur alone, while in other embodiments, combinations of mutations are present. In some embodiments, an altered Bacillus host cell strain that can be used to produce a lysozyme polypeptide of the invention is a Bacillus host strain that already includes a mutation in one or more of the above-mentioned genes. In addition, Bacillus sp. host cells that comprise mutation(s) and/or deletions of endogenous protease genes find use. In some embodiments, the Bacillus host cell comprises a deletion of the aprE and the nprE genes. In other embodiments, the Bacillus sp. host cell comprises a deletion of 5 protease genes, while in other embodiments, the Bacillus sp. host cell comprises a deletion of 9 protease genes (See e.g., US 2005/0202535, incorporated herein by reference).

Host cells are transformed with at least one nucleic acid encoding at least one lysozyme polypeptide of the invention using any suitable method known in the art. Methods for introducing a nucleic acid (e.g., DNA) into Bacillus cells or E. coli cells utilizing plasmid DNA constructs or vectors and transforming such plasmid DNA constructs or vectors into such cells are well known. In some embodiments, the plasmids are subsequently isolated from E. coli cells and transformed into Bacillus cells. However, it is not essential to use intervening microorganisms such as E. coli, and in some embodiments, a DNA construct or vector is directly introduced into a Bacillus host.

Suitable methods for introducing nucleic acid sequences of the invention into Bacillus cells include those described, for example, in Ferrari et al., “Genetics,” in Harwood et al. [eds.], Bacillus, Plenum Publishing Corp. [1989], pp. 57-72; Saunders et al., J. Bacteriol. 157:718-726 [1984]; Hoch et al., J. Bacteriol. 93:1925 -1937 [1967]; Mann et al., Current Microbiol. 13:131-135 [1986]; Holubova, Folia Microbiol. 30:97 [1985]; Chang et al., Mol. Gen. Genet. 168:11-115 [1979]; Vorobjeva et al., FEMS Microbiol. Lett. 7:261-263 [1980]; Smith et al., Appl. Env. Microbiol. 51:634 [1986]; Fisher et al., Arch. Microbiol. 139:213-217 [1981]; and McDonald, J. Gen. Microbiol. 130:203 [1984]). Indeed, such methods as transformation, including protoplast transformation and transfection, transduction, and protoplast fusion are well known and suited for use in the present invention. Methods known in the art to transform Bacillus cells include such methods as plasmid marker rescue transformation, which involves the uptake of a donor plasmid by competent cells carrying a partially homologous resident plasmid (See, Contente et al., Plasmid 2:555-571 [1979]; Haima et al., Mol. Gen. Genet. 223:185-191 [1990]; Weinrauch et al., J. Bacteriol. 154:1077-1087 [1983]; and Weinrauch et al., J. Bacteriol. 169:1205-1211 [1987]. In this method, the incoming donor plasmid recombines with the homologous region of the resident “helper” plasmid in a process that mimics chromosomal transformation.

In addition to commonly used methods, in some embodiments, host cells are directly transformed with a DNA construct or vector comprising a nucleic acid encoding a lysozyme polypeptide of the invention (i.e., an intermediate cell is not used to amplify, or otherwise process, the DNA construct or vector prior to introduction into the host cell). Introduction of the DNA construct or vector of the invention into the host cell includes those physical and chemical methods known in the art to introduce a nucleic acid sequence (e.g., DNA sequence) into a host cell without insertion into the host genome. Such methods include, but are not limited to calcium chloride precipitation, electroporation, naked DNA, liposomes and the like. In additional embodiments, DNA constructs or vector are co-transformed with a plasmid, without being inserted into the plasmid. In further embodiments, a selective marker is deleted from the altered Bacillus strain by methods known in the art (See, Stahl et al., J. Bacteriol. 158:411-418 [1984]; and Palmeros et al., Gene 247:255 -264 [2000]).

In some embodiments, the transformed cells of the present invention are cultured in conventional nutrient media. The suitable specific culture conditions, such as temperature, pH and the like are known to those skilled in the art and are well described in the scientific literature. In some embodiments, the invention provides a culture (e.g., cell culture) comprising at least one lysozyme polypeptide or at least one nucleic acid of the disclosure.

In some embodiments, host cells transformed with at least one polynucleotide sequence encoding at least one lysozyme polypeptide of the disclosure are cultured in a suitable nutrient medium under conditions permitting the expression of the present lysozyme, after which the resulting lysozyme is recovered from the culture. In some embodiments, the lysozyme produced by the cells is recovered from the culture medium by conventional procedures, including, but not limited to for example, separating the host cells from the medium by centrifugation or filtration, precipitating the proteinaceous components of the supernatant or filtrate by means of a salt (e.g., ammonium sulfate), chromatographic purification (e.g., ion exchange, gel filtration, affinity, etc.).

In some embodiments, a lysozyme polypeptide produced by a recombinant host cell is secreted into the culture medium. A nucleic acid sequence that encodes a purification facilitating domain may be used to facilitate purification of proteins. A vector or DNA construct comprising a polynucleotide sequence encoding a lysozyme polypeptide may further comprise a nucleic acid sequence encoding a purification facilitating domain to facilitate purification of the lysozyme polypeptide (See e.g., Kroll et al., DNA Cell Biol. 12:441-53 [1993]). Such purification facilitating domains include, but are not limited to, for example, metal chelating peptides such as histidine-tryptophan modules that allow purification on immobilized metals (See, Porath, Protein Expr. Purif. 3:263-281 [1992]), protein A domains that allow purification on immobilized immunoglobulin, and the domain utilized in the FLAGS extension/affinity purification system. The inclusion of a cleavable linker sequence such as Factor XA or enterokinase (e.g., sequences available from Invitrogen, San Diego, CA) between the purification domain and the heterologous protein also find use to facilitate purification.

Assays for detecting and measuring the enzymatic activity of an enzyme, such as a lysozyme polypeptide of the invention, are well known. Various assays for detecting and measuring activity of lysozymes, are also known to those of ordinary skill in the art. In particular, assays are available for measuring lysozyme activity such as those described in the examples, those described by Gorin et al (Gorin, G., Wang, S. F., Papapavlou, L., (1971) Assay of lysozyme by its lytic action on M-lysodeikticus cells. Analytical Biochemistry 39:113-127), or those available commercially such as in the EnzChek® Lysozyme Assay Kit (ThermoFisher).

A variety of methods can be used to determine the level of production of a mature lysozyme in a host cell. Such methods include, but are not limited to, for example, methods that utilize either polyclonal or monoclonal antibodies specific for the lysozyme. Exemplary methods include, but are not limited to enzyme-linked immunosorbent assays (ELISA), radioimmunoassays (RIA), fluorescent immunoassays (FIA), and fluorescent activated cell sorting (FACS). These and other assays are well known in the art (See e.g., Maddox et al., J. Exp. Med. 158:1211 [1983]).

In some other embodiments, the invention provides methods for making or producing a mature lysozyme polypeptide of the disclosure. A mature lysozyme polypeptide does not include a signal peptide or a propeptide sequence. Some methods comprise making or producing a lysozyme polypeptide of the disclosure in a recombinant bacterial host cell, such as for example, a Bacillus sp. cell (e.g., a B. subtilis cell). In some embodiments, the disclosure provides a method of producing a lysozyme polypeptide of the invention, the method comprising cultivating a recombinant host cell comprising a recombinant expression vector comprising a nucleic acid encoding a lysozyme polypeptide of the disclosure (e.g. a polypeptide having at least 50%, 55%, 60%, 65%, 70%, 75%, 80%, 85%, 90%, 95%, 96%, 97%, 98%, 99%, or 100% to an amino acid sequence of SEQ ID NOs: 1, 2, 3, 4, 5, 6, 7, 8, 9, 10, 11, 12, 13, 14, 15, 16, 17, 18, 19, 20, 21, 22, 23, 24, and 25) under conditions conducive to the production of the lysozyme polypeptide. Some such methods further comprise recovering the lysozyme polypeptide from the culture.

In some embodiments the disclosure provides methods of producing a lysozyme polypeptide of the invention, the methods comprising: (a) introducing a recombinant expression vector comprising a nucleic acid encoding a lysozyme polypeptide of the disclosure (e.g. a polypeptide having at least 50%, 55%, 60%, 65%, 70%, 75%, 80%, 85%, 90%, 95%, 96%, 97%, 98%, 99%, or 100% to an amino acid sequence of SEQ ID NOs: 1, 2, 3, 4, 5, 6, 7, 8, 9, 10, 11, 12, 13, 14, 15, 16, 17, 18, 19, 20, 21, 22, 23, 24, and 25) into a population of cells (e.g., bacterial cells, such as B. subtilis cells); and (b) culturing the cells in a culture medium under conditions conducive to produce the lysozyme polypeptide encoded by the expression vector. Some such methods further comprise: (c) isolating the lysozyme polypeptide from the cells or from the culture medium.

### Cleaning Methods

In one embodiment, methods for preventing, reducing or removing a biofilm or biofilm-related soiling are provided, where the methods comprise contacting the biofilm or biofilm-related soil with a polypeptide having lysozyme activity (e.g. a polypeptide having at least 50%, 55%, 60%, 65%, 70%, 75%, 80%, 85%, 90%, 95%, 96%, 97%, 98%, 99%, or 100% to an amino acid sequence of SEQ ID NOs: 1, 2, 3, 4, 5, 6, 7, 8, 9, 10, 11, 12, 13, 14, 15, 16, 17, 18, 19, 20, 21, 22, 23, 24, and 25) or a composition comprising a polypeptide having lysozyme activity.

In another embodiment, the disclosure provides a method for preventing, reducing or removing a biofilm from a textile or hard surface, where the method comprises contacting a textile or hard surface with a polypeptide having lysozyme activity, or a composition comprising a polypeptide having lysozyme activity, and optionally rinsing the textile or hard surface.

In one embodiment, the textile or hard surface comprises a biofilm, for example, on its surface. In one embodiment, the biofilm is reduced by at least about 10%, 15%, 20%, 25%, 30%, 35%, 40%, 45%, 50%, 55%, 60%, 65%, 70%, 75%, 80%, 85%, 90%, 95%, or more from the amount of the biofilm present on the surface or textile prior to contacting the surface or textile with the polypeptide having lysozyme activity or a composition comprising a polypeptide having lysozyme activity. In one embodiment, the level of reduction in the biofilm present on the surface or textile is assayed using a method available in the art to determine biofilm removal. In one embodiment, the biofilm level can be measured using the method provided in Examples 1, 3, 4, and 7 below.

In another embodiment, the prevention or reduction of a biofilm includes the reduction in the formation, growth, or proliferation of biofilm on a textile or hard surface. In one embodiment, the reduction in the formation, growth, or proliferation of biofilm on a textile or hard surface may be measured by following the change in the amount of the biofilm over a suitable time period with the method provided in Examples 1, 3, 4, and 7 below, or another suitable method in the art. For example, biofilm formation or growth may be inhibited in an amount ranging from 1% to about 99% relative to that of an untreated hard surface or textile. Biofilm formation may be inhibited by at least about 10%, at least about 20%, at least about 30%, at least about 40%, at least about 50%, at least about 60%, at least about 70%, at least about 80%, at least about 90%, or at least about 95% relative to biofilm formation on an untreated hard surface or textile. In another embodiment, the formation of biofilm on a surface may occur over a number of laundry cycles or may be delayed over a number of laundry cycles, compared to that of an untreated surface.

In another embodiment, methods for preventing, removing, or reducing microbes on a textile, on a surface, or in a solution are provided, where the methods comprise contacting the textile, surface, or solution with a polypeptide having lysozyme activity (e.g. a polypeptide having at least 50%, 55%, 60%, 65%, 70%, 75%, 80%, 85%, 90%, 95%, 96%, 97%, 98%, 99%, or 100% to an amino acid sequence of SEQ ID NOs: 1, 2, 3, 4, 5, 6, 7, 8, 9, 10, 11, 12, 13, 14, 16, 17, 18, 19, 20, 21, 22, 23, 24, and 25) or a composition comprising a polypeptide having lysozyme activity. In one embodiment, the reduction in microorganism count on a textile, on a surface, or in a solution may be measured using standard methods known in the art, such as by counting colonies, by measuring optical densities, or through the use of an indicator stain or dye. In one embodiment, the organisms prevented, removed, or reduced on a textile, on a surface, or in a solution may include, but are not limited to, one or more bacterial genera of Acinetobacter sp, Aeromicrobium sp, Brevundimonas sp., Burkholderia sp., Campylobacter sp., Clostridium sp., Desulfovibrio sp., Escherichia sp., Haemophilus sp., Lacobacillus sp., Lactococcus sp., Listeria sp., Microbacterium sp., Micrococcus sp. (e.g. Micrococcus luteus), Moraxella sp. (e.g. Moraxella osloensis), Porphyromonas sp., Pseudomonas sp. (e.g. Pseudomonas fluorescens, Pseudomonas putida, Pseudomonas aeruginosa), Salmonella sp., Staphylococcus sp. (e.g. Staphylococcus epidermidis, Staphylococcus aureus), Stenotrophomonas sp., Streptomyces sp., Streptococcus sp. (e.g. Streptococcus mutans), and Vibrio sp. In one embodiment, the organisms may include fungal or yeast species, such as Candida albicans.

In another embodiment, methods for preventing, removing, or reducing malodor on a textile, on a surface, or in a solution are provided, where the methods comprise contacting the textile, surface, or solution with a polypeptide having lysozyme activity (e.g. a polypeptide having at least 50%, 55%, 60%, 65%, 70%, 75%, 80%, 85%, 90%, 95%, 96%, 97%, 98%, 99%, or 100% to an amino acid sequence of SEQ ID NOs: 1, 2, 3, 4, 5, 6, 7, 8, 9, 10, 11, 12, 13, 14, 16, 17, 18, 19, 20, 21, 22, 23, 24, and 25) or a composition comprising a polypeptide having lysozyme activity. In one embodiment, reduction in malodor may be measured by human sensory observations, such as by smelling the surface, textile or solution, or by analytical measurement of malodorous compounds, such as but not limited to gas chromatography-mass spectrometry (GC/MS) or gas chromatography mass spectrometry with solid phase microextraction (GC/MS-SPME) or gas chromatography-olfactometry (GC-O). In one embodiment, malodor may be reduced by at least 5%, or at least 10%, or at least 20%, or at least 30%, or at least 40%, or at least 50%, or at least 60%, or at least 70%, or at least 80%, or at least 90% relative to untreated controls. The textile or surface can be contacted with the polypeptide or a composition comprising the polypeptide having lysozyme activity in a washing machine or in a manual wash tub (e.g. for handwashing). In one embodiment, the textile or surface is contacted with the polypeptide having lysozyme activity or the composition comprising a peptide having lysozyme activity in a wash liquor. In another embodiment, a solution containing the polypeptide having lysozyme activity is incubated with or flowed over the hard surface, such as by pumping the solution through tubing or pipes or by filling a reservoir with the solution.

In some embodiments, the textiles or surfaces are contacted with the polypeptide or compositions comprising the polypeptide under conditions for any amount of time desired or for any period of time sufficient to prevent, reduce or remove biofilm from the textile. In one embodiment, the contacting step is between about 5 minutes and about 10 days. In some embodiments, the contacting takes place in a wash liquor for about 5 to about 400 minutes, between about 5 minutes to about 300 minutes, between about 5 minutes to about 250 minutes, between about 5 minutes to about 200 minutes, between about 5 minutes to about 150 minutes, between about 5 minutes to about 100 minutes, between about 5 minutes to about 50 minutes, between about 5 minutes to about 30 minutes. In some embodiments, the prevention, reduction, or removal of biofilm occurs over many wash cycles, such that the total contacting time over multiple wash cycles is about 5 to about 400 minutes, between about 5 minutes to about 300 minutes, between about 5 minutes to about 250 minutes, between about 5 minutes to about 200 minutes, between about 5 minutes to about 150 minutes, between about 5 minutes to about 100 minutes, between about 5 minutes to about 50 minutes, between about 5 minutes to about 30 minutes.

In some embodiments, the textiles or articles are contacted with the polypeptide or compositions comprising the polypeptide under conditions having a temperature that allows for microorganism or biofilm prevention, reduction or removal from the textile or article. In some embodiments, the temperature in the methods disclosed herein include those between 10° to 60° C., between 10° to about 45° C., between 15° to about 55° C., between 15° to about 50° C., between 15° to about 45° C., between 20° to about 60° C., between 20° to about 50° C. and between 20° to about 45° C.

The polypeptides, compositions, and methods provided herein have utility in a wide array of applications in which preventing, reducing, or removing microorganisms or biofilms is desired, such as household cleaning, including in washing machines, dishwashers, and on household surfaces. The polypeptides, compositions, and methods also have applications in treating medical and dental biofilms, including but not limited to plaque on teeth, lung infections (e.g. Pulmozyme®), on catheters and implanted medical devices, on contact lenses, in medical instrument cleaning, and in wound dressings. The polypeptides, compositions, and methods also have applications as antimicrobials for personal care, including but not limited to in toothpaste, mouthwash, breath fresheners, cosmetics, creams, washes, rinses, wipes, toiletries, shampoos, and soaps. The polypeptides, compositions, and methods also have applications as antimicrobials for food and beverages, including but not limited to in vegetables, fruit, meat, poultry, fish, or packaging materials including sheets, bottles, vials, bags, boxes, trays, or cartons. The polypeptides, compositions, and methods provided herein can also be used to treat biofouling and microbial contamination in various industrial settings, including but not limited to industrial process water, wastewater treatment, cooling systems, evaporative condensers, fountains, filtration systems, ultrafiltration systems, heat exchangers, pulp and paper processing fluids, textile processing or products, printing fluids, metalworking fluids, hydraulic fluids, oilfield fluids, injection water, fracture fluids, drilling muds, holding tanks, fuel, petroleum processing fluids, gaskets, pipes, tubing, medical devices, water treatment facilities, marine equipment, animal care water and food delivery systems, holding pens, cages, barns, sheds, or floors. The polypeptides, compositions, and methods also have applications as antimicrobials for industrial cleaners, floor polishes, wood, wood products, leather, insulation, paints and coatings, fabrics, adhesives, bathroom and kitchen cleaners or wipes,

Another embodiment is directed to a method of laundering a textile, where the method comprises contacting a textile with a polypeptide having lysozyme activity, or a composition comprising a polypeptide having lysozyme activity for an amount of time sufficient to prevent, reduce or remove microbes and/or biofilm from the textile and optionally rinsing the textile.

Another embodiment is directed to a method for cleaning an article, where the method comprises contacting the article with a polypeptide having lysozyme activity or a composition having a polypeptide having lysozyme activity under conditions sufficient to reduce or remove microorganisms or a biofilm from the article, and optionally rinsing the article.

### Compositions

In one embodiment, the disclosure provides compositions (e.g. detergent compositions) for use in the methods provided herein. The compositions generally comprise a polypeptide having lysozyme activity (e.g. a polypeptide having at least 50%, 55%, 60%, 65%, 70%, 75%, 80%, 85%, 90%, 95%, 96%, 97%, 98%, 99%, or 100% to an amino acid sequence of SEQ ID NOs: 1, 2, 3, 4, 5, 6, 7, 8, 9, 10, 11, 12, 13, 14, 15, 16, 17, 18, 19, 20, 21, 22, 23, 24, and and one or more additional detergent components, such as a surfactant.

The compositions having a polypeptide having lysozyme activity, which find use in the methods provided herein, may comprise a polypeptide having lysozyme activity at a concentration in use of 0.001 to 10,000 mg/L, or 0.001 to 200mg/L, or 0.01 to 5000 mg/L, or to 2000 mg/L, or 0.01 to 1300 mg/L, or 0.01 to 500 mg/L, or 0.1 to 5000 mg/L, or 0.1 to 2000 mg/L, or 0,1 to 1300 mg/L, or 0.1 to 100 mg/L, or 0.1 to 50 mg/L, or 1 to 5000 mg/L, or 1 to 1300 mg/L, or 1 to 500 mg/L, or 1 to 100 mg/L, or 10 to 5000 mg/L, or 10 to 1300 mg/L, or to 500 mg/L. In another embodiment, the composition may contain a polypeptide having lysozyme activity in an amount of 0.002 to 5000 mg of protein, such as 0.005 to 1300 mg of protein, or 0.01 to 5000 mg of protein, or 0.01 to 1300 mg of protein, or 0.1 to 500( )mg of protein, or 1 to 1300 mg of protein, preferably 0.1 to 1300 mg, of protein, more preferably 1 to 1300 mg of protein, even more preferably 10 to 500 mg of protein, per liter of wash liquor, or in the amount of at least 0.002 ppm active lysozyme. In another embodiment, the detergent composition comprises a polypeptide having lysozyme activity in an amount to provide the lysozyme in a wash liquor in an amount of between 0,01 to 1000 ppm, between about 0.1 to 5000 ppm, between about 0.1 to 2500 ppm, between about 0.1 to 1500 ppm, between about 0.1 to 1300 ppm, between about 0.1 to 1000 ppm, between about 0.1 to 500 ppm, between 1 to 1300 ppm, between about 1 to about 500 ppm, between about 1 to about 100 ppm, between 10 to 1300 ppm, between about 10 and 500 PPM, between about 50 and 1300 ppm, between about 50 and 500 ppm in the wash liquor.

In some embodiments, the lysozyme for use herein includes those lysozyme polypeptides described in WO2018/113745, WO2018/206001, WO2018/127532, WO2018/113745, WO2018/113743, WO2013/072305, WO2013/076253, WO2012/035103, WO2011/104339, WO2017/000922, and U.S. Pat. No. 9609876. In some embodiments, the lysozyme for use herein includes commonly known lysozymes including hen egg white lysozyme and/or T4 lysozyme.

In one embodiment, the composition comprises a lysozyme and at least one additional detergent component, and optionally one or more additional enzymes.

Also provided are detergent compositions for use in the methods provided herein. As used herein, the term “detergent composition” or “detergent formulation” is used in reference to a composition intended for use in a wash medium (e.g. a wash liquor) for the cleaning of soiled or dirty objects, including particular textile or non-textile objects or items. Such compositions of the present invention are not limited to any particular detergent composition or formulation. Indeed, in some embodiments, the detergents of the invention comprise at least one lysozyme polypeptide (a polypeptide having at least 50%, 55%, 60%, 65%, 70%, 75%, 80%, 85%, 90%, 95%, 96%, 97%, 98%, 99%, or 100% to an amino acid sequence of SEQ ID NOs: 1, 2, 3, 4, 5, 6, 7, 8, 9, 10, 11, 12, 13, 14, 15, 16, 17, 18, 19, 20, 21, 22, 23, 24, and 25) and, in addition, one or more surfactants, transferase(s), hydrolytic enzymes, oxido reductases, builders (e.g., a builder salt), bleaching agents, bleach activators, bluing agents, fluorescent dyes, caking inhibitors, masking agents, enzyme activators, antioxidants, and/or solubilizers. In some instances, a builder salt is a mixture of a silicate salt and a phosphate salt, preferably with more silicate (e.g., sodium metasilicate) than phosphate (e.g., sodium tripolyphosphate). Some compositions of the invention, such as, but not limited to, cleaning compositions or detergent compositions, do not contain any phosphate (e.g., phosphate salt or phosphate builder).

In some embodiments, the cleaning or detergent compositions of the present disclosure further comprise adjunct materials including, but not limited to, surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See e.g., U.S. Pat. Nos. 6,610,642, 6,605,458, 5,705,464, 5,710,115, 5,698,504, 5,695,679, 5,686,014 and 5,646,101, all of which are incorporated herein by reference).

The detergent or cleaning compositions of the present invention are advantageously employed for example, in laundry applications, hard surface cleaning, dishwashing applications, as well as personal care or cosmetic applications such as dentures, toothpastes, cosmetics, lotions, shampoos, conditioners, creams, wipes, pre-moistened wipes, balms, pastes, or ointments. In addition, due to the unique advantages of increased effectiveness in lower temperature solutions, the enzymes of the present invention are ideally suited for laundry applications. Furthermore, the enzymes of the present invention find use in granular and liquid compositions.

Enzyme component weights are based on total active protein. All percentages and ratios are calculated by weight unless otherwise indicated. All percentages and ratios are calculated based on the total composition unless otherwise indicated. In laundry detergent compositions, the enzyme levels are expressed in ppm, which equals mg active protein/kg detergent composition.

In some embodiments, the laundry detergent compositions described herein further comprise a surfactant. In some embodiments, the surfactant is selected from a non-ionic, ampholytic, semi-polar, anionic, cationic, zwitterionic, and combinations and mixtures thereof. In yet a further embodiment, the surfactant is selected from an anionic surfactant, a cationic surfactant, a zwitterionic surfactant, and combinations thereof. In some embodiments, the laundry detergent compositions described herein comprise from about 0.1% to about 60%, about 1% to about 50%, or about 5% to about 40% surfactant by weight of the composition.

Exemplary surfactants include, but are not limited to sodium dodecylbenzene sulfonate, C12-14 pareth-7, C12-15 pareth-7, sodium C12-15 pareth sulfate, C14-15 pareth-4, sodium laureth sulfate (e.g., Steol CS-370), sodium hydrogenated cocoate, C12 ethoxylates (Alfbnic 1012-6, Hetoxol LA7, Hetoxol LA4), sodium alkyl benzene sulfonates (e.g., Nacconol and combinations and mixtures thereof. Anionic surfactants include but are not limited to linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. Nonionic surfactants include but are not limited to alcohol ethoxylate (AEO or AE), carboxylated alcohol ethoxylates, nonylphenol ethoxylate, alkylpolyglycoside, alkyldimethylamine oxide, ethoxylated fatty acid monoethanolamide, fatty acid monoetha.nolamide, polyhydroxy alkyl fatty acid amide (e.g., as described in WO92/06154), polyoxyethylene esters of fatty acids, polyoxyethylene sorbitan esters (e.g., TWEENs), polyoxyethylene alcohols, polyoxyethylene isoalcohols, polyoxyethylene ethers (e.g., TRITONs and BRIJ), polyoxyethylene esters, polyoxyethylene-p- tert-octylphenols or octylphenyl-ethylene oxide condensates (e.g., NONIDET P40), ethylene oxide condensates with fatty alcohols (e.g., LUBROL), polyoxyethylene nonylphenols, polyalkylene glycols (SYNPERONIC F108), sugar-based surfactants (e.g., glycopyranosides, thioglycopyranosides), and combinations and mixtures thereof.

In a further embodiment, the laundry detergent compositions described herein further comprise a surfactant mixture that includes, but is not limited to 5-15% anionic surfactants, <5% nonionic surfactants, cationic surfactants, phosphonates, soap, enzymes, perfume, butylphenyl methyl propionate, geraniol, zeolite, polycarboxylates, hexyl cinnamal, limonene, cationic surfactants, citronellol, and benzisothiazolinone.

The laundry detergent compositions described herein may additionally include one or more detergent builders or builder systems, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, an optical brightener, a fabric conditioner, and a perfume. The laundry detergent compositions described herein may also include additional enzymes selected from proteases, amylases, cellulases, lipases, hexosaminidases, mannanases, nucleases, pectinases, xyloglucana.ses, or perhydrolases, as provided in more detail herein.

In some embodiments, the laundry detergent compositions described herein further comprises from about 1%, from about 3% to about 60% or even from about 5% to about 40% builder by weight of the cleaning composition. Builders may include, but are not limited to, the alkali metals, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxy benzene-2,4.6-trisulphonic acid, and carboxymethyloxysuccinic acid, the various alkali metals, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof

In some embodiments, the builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and. potassium tripolyphosphate, etc.). Any suitable builder can find use in the compositions described herein, including those known in the art.

In some embodiments, the laundry detergent compositions described herein further comprise an adjunct ingredient including, but not limited to surfactants, builders, bleaches, bleach activators, bleach catalysts, additional enzymes, an enzyme stabilizer (including, for example, an enzyme stabilizing system), chelants, optical brighteners, soil release polymers, dye transfer agents, dye transfer inhibiting agents, catalytic materials, hydrogen peroxide, sources of hydrogen peroxide, preformed peracids, polymeric dispersing agents, clay soil removal agents, structure elasticizing agents, dispersants, suds suppressors, dyes, perfumes, colorants, filler salts, hydrotropes, photoactivators, fluorescers, fabric conditioners, hydrolyzable surfactants, solvents, preservatives, anti-oxidants, anti-shrinkage agents, anti-wrinkle agents, germicides, fungicides, color speckles, anti-corrosion agents, alkalinity sources, solubilizing agents, carriers, processing aids, pigments, pH control agents, and combinations thereof. (See, e.g., U.S. Pat. Nos. 6,610,642, 6,605,458, 5,705,464, 5,710,115, 5,698,504, , 5,695,679, 5,686,014, and 5,646,101), In some embodiments, one or more adjunct is incorporated for example, to assist or enhance cleaning performance, for treatment of the substrate to be cleaned, or to modify the aesthetics of the cleaning composition as is the case with perfumes, colorants, dyes or the like. Any such adjunct ingredient is in addition to the lysozyme polypeptide described herein. In some embodiments, the adjunct ingredient is selected from surfactants, enzyme stabilizers, builder compounds, polymeric compounds, bleaching agents, additional enzymes, suds suppressors, dispersants, lime-soap dispersants, soil suspension agents, softening agents, anti-redeposition agents, corrosion inhibitors, and combinations thereof.

In some further embodiments, the laundry detergent compositions described herein comprise one or more enzyme stabilizer. In some embodiments, the enzyme stabilizer is a water-soluble source of calcium and/or magnesium ions. In some embodiments, the enzyme stabilizers include oligosaccharides, polysaccharides, and inorganic divalent metal salts, including alkaline earth metals, such as calcium salts. In some embodiments, the enzymes employed herein are stabilized by the presence of water-soluble sources of zinc (II), calcium (II) and/or magnesium (H) ions in the finished compositions that provide such ions to the enzymes, as well as other metal ions (e.g, barium (II), scandium (II), iron (II), manganese (II), aluminum (III), tin (II), cobalt (II), copper (II), nickel (II), and oxovanadium (IV)). Chlorides and sulfates also find use in some embodiments. Exemplary oligosaccharides and polysaccharides (e.g., dextrins) are described, for example, in WO07145964. In some embodiments, the laundry detergent compositions described herein contain reversible enzyme inhibitors such as, for example, glycoside or protein lysozyme inhibitors, boron-containing compounds (e.g., borate, 4-formyl phenyl boronic acid, and phenyl-boronic acid derivatives, such as, e.g., are described in WO9641859), a peptide aldehydes (such as, e.g., is described in WO2009118375 and WO2013004636), or combinations thereof.

The cleaning compositions herein are typically formulated such that, during use in aqueous cleaning operations, the wash water will have a pH of from about 3.0 to about 11. Liquid product formulations are typically formulated to have a neat pH from about 5,0 to about 9.0. Granular laundry products are typically formulated to have a pH from about 8.0 to about 11.0. Techniques for controlling pH at recommended usage levels include the use of buffers, alkalis, acids, etc., and are well known to those skilled in the art.

Suitable high pH cleaning compositions typically have a neat pH of from about 9.0 to about 11.0, or even a neat pH of from 9.5 to 10.5. Such cleaning compositions typically comprise a sufficient amount of a pH modifier, such as sodium hydroxide, monoethanolamine, or hydrochloric acid, to provide such cleaning composition with a neat pH of from about 9.0 to about 11.0. Such compositions typically comprise at least one base-stable enzyme. In some embodiments, the compositions are liquids, while in other embodiments, they are solids.

Concentrations of detergent compositions in typical wash solutions throughout the world vary from less than about 800 ppm of detergent composition (“low detergent concentration geographies”), for example about 667 ppm in Japan, to between about 800 ppm to about 2000 ppm (“medium detergent concentration geographies”), for example about 975 ppm in U.S. and about 1500 ppm in Brazil, to greater than about 2000 ppm (“high detergent concentration geographies”), for example about 4500 ppm to about 5000 ppm in Europe and about 6000 ppm in high suds phosphate builder geographies.

In some embodiments, the detergent compositions described herein may be utilized at a temperature of from about 10° C. to about 60° C., or from about 20° C. to about 60° C., or from about 30° C. to about 60° C., from about 40° C. to about 60° C., from about 40° C. to about 55° C., or all ranges within 10° C. to 60° C. In some embodiments, the detergent compositions described herein are used in “cold water washing” at temperatures of from about 10° C. to about 40° C., or from about 20° C. to about 30° C., from about 15° C. to about 25° C., from about 15° C. to about 35° C., or all ranges within 10° C. to 40° C.

As a further example, different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60-120 ppm) to hard (121-181 ppm) water has 60 to 181 parts per million (parts per million converted to grains per U.S. gallon is ppm # divided by 17.1 equals grains per gallon) of hardness minerals.

TABLE I
Water Hardness Levels

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (for example about 10.5 to about 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, for example about 3 grains per gallon mixed Ca²⁺/Mg²⁺.

In other embodiments, the composition described herein may further comprise one or more additional enzyme. The one or more additional enzyme is selected from acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, additional proteases, pullulanases, reductases, rhamnogalacturonases, beta-glucanases, tannases, transglutaminases, xylan acetyl-esterases, xylanases, xyloglucanases, xylosidases, and any combination or mixture thereof. Some embodiments are directed to a combination of enzymes (i.e., a “cocktail”) comprising enzymes like amylase, protease, lipase, mannanase, and/or nuclease in conjunction with one or more lysozyme polypeptides in the compositions provided herein.

In some embodiments, the compositions provided herein comprise a polypeptide having lysozyme activity in combination with one or more protease. The protease for use in combination with the lysozyme in the compositions of the instant disclosure include any polypeptide having protease activity. In one embodiment, the additional protease is a serine protease. In another embodiment, the additional protease is an additional metalloprotease, a fungal subtilisin, or an alkaline microbial protease or a trypsin-like protease. Suitable additional proteases include those of animal, vegetable or microbial origin. In some embodiments, the protease is a microbial protease. In other embodiments, the protease is a chemically or genetically modified mutant. In another embodiment, the protease is subtilisin like protease or a trypsin-like protease. Exemplary subtilisin proteases include those derived from for example, Bacillus (e.g., e.g., BPN′, Carlsberg, subtilisin 309, subtilisin 147, TY145, and subtilisin 168), or fungal origin, such as, for example, those described in U.S. Pat. No. 8,362,222. Exemplary additional proteases include but are not limited to those described in WO92/21760, WO92/17577, WO95/23221, WO2008/010925, WO09/149200, WO09/149144, WO09/149145, WO 10/056640, WO10/056653, WO2010/0566356, WO11/072099, WO2011/13022, WO11/140364, WO 12/151534, WO2015/038792, WO2015/089447, WO2015/089441, WO2016/097352, WO 2017/215925, US Publ. No. 2008/0090747, U.S. Pat. Nos. 5,801,039, 5,340,735, 5,500,364, 5,855,625, 34,606, 5,955,340, 5,700,676 6,312,936, 6,482,628, 8,530,219, U.S. Provisional Appl Nos. 62/180673 and 62/161077, and PCT Appl Nos. PCT/U52015/021813, PCT/U52015/055900, PCT/U52015/057497, PCT/U52015/057492, PCT/U52015/057512, PCT/U52015/057526, PCT/U52015/057520, PCT/U52015/057502, PCT/US2016/022282, and PCT/US16/32514, as well as metalloproteases described in WO1999014341, WO1999033960, WO1999014342, WO1999034003, WO2007044993, WO2009058303, WO 2009058661, WO2014071410, WO2014194032, WO2014194034, WO 2014194054, and WO 2014/194117. Exemplary additional proteases include, but are not limited to trypsin (e.g., of porcine or bovine origin) and the Fusarium protease described in WO89/06270. Exemplary commercial proteases include, but are not limited to MAXATASE®, MAXACAL™, MAXAPEM™, OPTICLEAN®, OPTIMASE®, PROPERASE®, PURAFECT®, PURAFECT® OXP, PURAIVIAX™, EXCELLASE™, PREFERENZ™ proteases (e.g. P100, P110, P280, P300), EFFECTENZ™ proteases (e.g. P1000, P1050, P2000), EXCELLENZ™ proteases (e.g. P1000), ULTIMASE®, and PURAFAST™ (DuPont); ALCALASE®, BLAZE®, and BLAZE® variants, BLAZE® EVITY® 16L, CORONASE®, SAVINASE®, SAVINASE® ULTRA, SAVINASE® EVITY®, SAVINASE® EVERTS®, PRIMASE®, DURAZYM™, POLARZYME®, OVOZYME®, KANNASE®, LIQUANASE®, LIQUANASE EVERTS®, NEUTRASE®, RELASE®, PROGRESS UNO®, EASYZYME®, and ESPERASE® (Novozymes); BLAP™ and BLAP™ variants (Henkel); KAP (B. alkalophilus subtilisin (Kao)); and BIOTOUCH® (AB Enzymes). Exemplary metalloproteases include nprE, the recombinant form of neutral metalloprotease expressed in B. subtilis (See e.g., WO 07/044993), and PMN, the purified neutral metalloprotease from B. amyloliquefaciens.

In some embodiments, the compositions provided herein comprise a polypeptide having lysozyme activity in combination with one or more amylases. In one embodiment, the composition comprises from about 0.00001% to about 10%, about 0.0001% to about 10%, about to about 5%, about 0.001% to about 2%, or about 0.005% to about 0.5% amylase by weight composition. Any amylase (e.g., alpha and/or beta) suitable for use in alkaline solutions may be useful to include in such composition. An exemplary amylase can be a chemically or genetically modified mutant. Exemplary amylases include, but are not limited to those of bacterial or fungal origin, such as, for example, amylases described in GB 1,296,839, WO9100353, WO9402597, WO94183314, WO9510603, WO9526397, WO9535382, WO9605295, WO9623873, WO9623874, WO 9630481, WO9710342, WO9741213, WO9743424, WO9813481, WO 9826078, WO9902702, WO 9909183, WO9919467, WO9923211, WO9929876, WO9942567, WO 9943793, WO9943794, WO 9946399, WO0029560, WO0060058, WO0060059, WO0060060, WO 0114532, WO0134784, WO 0164852, WO0166712, WO0188107, WO0196537, WO02092797, WO 0210355, WO0231124, WO 2004055178, WO2004113551, WO2005001064, WO2005003311, WO 2005018336, WO2005019443, WO2005066338, WO2006002643, WO2006012899, WO2006012902, WO2006031554, WO 2006063594, WO2006066594, WO2006066596, WO2006136161, WO 2008000825, WO2008088493, WO2008092919, WO2008101894, WO2008/112459, WO2009061380, WO2009061381, WO 2009100102, WO2009140504, WO2009149419, WO 2010/059413, WO 2010088447, WO2010091221, WO2010104675, WO2010115021, WO10115028, WO2010117511, WO 2011076123, WO2011076897, WO2011080352, WO2011080353, WO 2011080354, WO2011082425, WO2011082429, WO 2011087836, WO2011098531, WO2013063460, WO2013184577, WO 2014099523, WO2014164777, and WO2015077126. Exemplary commercial amylases include, but are not limited to AMPLIFY®, DURAMYL®, TERMAMYL®, FUNGAMYL®, STAINZYME®, STAINZYME PLUS®, STAINZYME PLUS®, STAINZYME ULTRA® EVITY®, and BAN™ (Novozymes); EFFECTENZ™ S 1000, POWERASE™, PREFERENZ™ S 100, PREFERENZ™ S 110, EXCELLENZ™ S 2000, RAPIDASE® and MAXAMYL® P (DuPont).

In some embodiments, the compositions provided herein comprise a polypeptide having lysozyme activity in combination with one or more lipases. In some embodiments, the composition comprises from about 0.00001% to about 10%, about 0.0001% to about 10%, about to about 5%, about 0.001% to about 2%, or about 0.005% to about 0.5% lipase by weight composition. An exemplary lipase can be a chemically or genetically modified mutant. Exemplary lipases include, but are not limited to, e.g., those of bacterial or fungal origin, such as, e.g., H. lanuginosa lipase (see, e.g., EP 258068 and EP 305216), T. lanuginosa lipase (see, e.g., WO 2014/059360 and WO2015/010009), Rhizomucor miehei lipase (see, e.g., EP 238023), Candida lipase, such as C. antarctica lipase (e.g., C. antarctica lipase A or B) (see, e.g., EP 214761), Pseudomonas lipases such as P. alcaligenes and P. pseudoalcaligenes lipase (see, e.g., EP 218272), P. cepacia lipase (see, e.g., EP 331376), P. stutzeri lipase (see, e.g., GB 1,372,034), P. fluorescens lipase, Bacillus lipase (e.g., B. subtilis lipase (Dartois et al., Biochem. Biophys. Acta 1131:253-260 (1993)), B. stearothermophilus lipase (see, e.g., JP 64/744992), and B. pumilus lipase (see, e.g., WO 91/16422)). Exemplary cloned lipases include, but are not limited to Penicillium camembertii lipase (See, Yamaguchi et al., Gene 103:61-67 (1991)), Geotrichum candidum lipase (See, Schimada et al., J. Biochem., 106:383-388 (1989)), and various Rhizopus lipases, such as, R. delemar lipase (See, Hass et al., Gene 109:117-113 (1991)), R. niveus lipase (Kugimiya et al., Biosci. Biotech. Biochem. 56:716-719 (1992)) and R. oryzae lipase. Other lipolytic enzymes, such as cutinases, may also find use in one or more composition described herein, including, but not limited to, e.g., cutinase derived from Pseudomonas mendocina (see, WO 88/09367) and/or Fusarium solani pisi (see, WO90/09446). Exemplary commercial lipases include, but are not limited to M1 LIPASE™, LUMA FAST™, and LIPOMAX™ (DuPont); LIPEX®, LIPOCLEAN®, LIPOLASE® and LIPOLASE® ULTRA (Novozymes); and LIPASE P™ (Amano Pharmaceutical Co. Ltd).

In some embodiments, the compositions provided herein comprise a polypeptide having lysozyme activity in combination with one or more mannanases. In one embodiment, the composition comprises from about 0.00001% to about 10%, about 0.0001% to about 10%, about to about 5%, about 0.001% to about 2%, or about 0.005% to about 0.5% mannanase by weight composition. An exemplary mannanase can be a chemically or genetically modified mutant. Exemplary mannanases include, but are not limited to, those of bacterial or fungal origin, such as, for example, those described in WO 2016/007929; WO2017/079756, WO2017/079751, U.S. Pat. Nos. 6,566,114; 6,602,842; and 6,440,991. Exemplary commercial mannanases include, but are not limited to MANNAWAY® (Novozymes) and EFFECTENZ™ M 1000, EFFECTENZ™ M 2000, PREFERENZ® M 100, MANNASTAR®, and PURABRITE™ (DuPont). Exemplary combinations of mannanases that can be combined in the compositions provided herein include those described in WO2019/081515.

In some embodiments, the compositions and methods provided herein comprise a polypeptide having lysozyme activity in combination with a nuclease, such as a DNase or RNase. Exemplary nucleases include, but are not limited to, those described in WO2015181287, WO2015155350, WO2016162556, WO2017162836, WO2017060475 (e.g. SEQ ID NO: 21), WO2018184816, WO2018177936, WO2018177938, WO2018/185269, WO2018185285, WO2018177203, WO2018184817, WO2019084349, WO2019084350, WO2019081721, WO2018076800, WO2018185267, WO2018185280, WO2018206553, and WO2019/086530. Other nucleases which can be used in combination with the polypeptides having lysozyme activity in the compositions and methods provided herein include those described in Nijland R, Hall M J, Burgess J G (2010) Dispersal of Biofilms by Secreted, Matrix Degrading, Bacterial DNase. PLoS ONE 5(12) and Whitchurch, C. B., Tolker-Nielsen, T., Ragas, P. C., Mattick, J. S. (2002) Extracellular DNA required for bacterial biofilm formation. Science 295: 1487.

Yet a still further embodiment is directed to a composition comprising one or more lysozymes described herein and one or more cellulase. In one embodiment, the composition comprises from about 0.00001% to about 10%, 0.0001% to about 10%, about 0.001% to about 5%, about 0.001% to about 2%, or about 0.005% to about 0.5% cellulase by weight of composition. Any suitable cellulase may find use in a composition described herein. An exemplary cellulase can be a chemically or genetically modified mutant. Exemplary cellulases include but are not limited, to those of bacterial or fungal origin, such as, for example, those described in WO2005054475, WO2005056787, U.S. Pat. No. 7,449,318, 7,833,773, 4,435,307; EP 0495257; and US Provisional Appl. No. 62/296,678. Exemplary commercial cellulases include, but are not limited to, CELLUCLEAN®, CELLUZYME®, CAREZYME®, ENDOLASE®, RENOZYME®, and CAREZYME® PREMIUM (Novozymes); REVITALENZ™ 100, REVITALENZ™ 200/220, and REVITALENZ® 2000 (DuPont); and KAC-500(B)™ (Kao Corporation). In some embodiments, cellulases are incorporated as portions or fragments of mature wild-type or variant cellulases, wherein a portion of the N-terminus is deleted (see, e.g., U.S. Pat. No. 5,874,276).

In some embodiments, the laundry detergent compositions described herein comprise at least one chelating agent. Suitable chelating agents may include, but are not limited to copper, iron, and/or manganese chelating agents, and mixtures thereof. In some embodiments, the laundry detergent compositions described herein comprises from about 0.1% to about 15% or even from about 3.0% to about 10% chelating agent by weight of composition.

In some still further embodiments, the laundry detergent compositions described herein comprise at least one deposition aid. Suitable deposition aids include, but are not limited to, polyethylene glycol, polypropylene glycol, polycarboxylate, soil release polymers such as polyterephthalic acid, clays such as kaolinite, montmorillonite, attapulgite, illite, bentonite, halloysite, and mixtures thereof.

In some embodiments, the laundry detergent compositions described herein comprise at least one anti-redeposition agent.

In some embodiments, the laundry detergent compositions described herein comprise one or more dye transfer inhibiting agent. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones, and polyvinylimidazoles, or mixtures thereof. In some embodiments, the laundry detergent compositions described herein comprise from about 0.0001% to about 10%, from about 0.01% to about 5%, or even from about 0.1% to about 3% dye transfer inhibiting agent by weight of composition.

In some embodiments, the laundry detergent compositions described herein comprise one or more silicates. In some such embodiments, sodium silicates (e.g., sodium disilicate, sodium metasilicate, and crystalline phyllosilicates) find use. In some embodiments, the laundry detergent compositions described herein comprise from about 1% to about 20% or from about 5% to about 15% silicate by weight of the composition.

In yet further embodiments, the laundry detergent compositions described herein comprise one or more dispersant. Suitable water-soluble organic materials include, but are not limited to the homo- or co-polymeric acids or their salts, in which the polycarboxylic acid comprises at least two carboxyl radicals separated from each other by not more than two carbon atoms.

In some embodiments, the laundry detergent compositions described herein comprise one or more bleach, bleach activator, and/or bleach catalyst. In some embodiments, the laundry detergent compositions described herein comprise inorganic and/or organic bleaching compound(s). Inorganic bleaches may include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Suitable salts include, for example, those described in EP2100949. Bleach activators are typically organic peracid precursors that enhance the bleaching action in the course of cleaning at temperatures of 60° C. and below. Bleach activators suitable for use herein include compounds which, under perhydrolysis conditions, give aliphatic peroxycarboxylic acids having preferably from about 1 to about 10 carbon atoms, in particular from about 2 to about 4 carbon atoms, and/or optionally substituted perbenzoic acid. Bleach catalysts typically include, for example, manganese triazacyclononane and related complexes, and cobalt, copper, manganese, and iron complexes, as well as those described in U.S. Pat. Nos. 4,246,612, 5,227,084, 4,810,410, WO9906521, and EP2100949. In some embodiments, a lysozyme is included in combination with an enzymatic catalyst for generation of peracids such as peracetic acid, such as those described in WO2005/056782A2, U.S. Pat. No. 7,754,460 B2, US2008/0176299, or US2006/0286651.

In some embodiments, the laundry detergent compositions described herein comprise one or more catalytic metal complex. In some embodiments, a metal-containing bleach catalyst finds use. In other embodiments, the metal bleach catalyst comprises a catalyst system comprising a transition metal cation of defined bleach catalytic activity (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (See, e.g., U.S. Pat. No. 4,430,243). In some embodiments, the laundry detergent compositions described herein are catalyzed by means of a manganese compound. Such compounds and levels of use are well known in the art (See, e.g., U.S. Pat. No. 5,576,282). In additional embodiments, cobalt bleach catalysts find use in the laundry detergent compositions described herein. Various cobalt bleach catalysts are known in the art (See, e.g., U.S. Pat. Nos. 5,597,936 and 5,595,967) and are readily prepared by known procedures.

Some embodiments are directed to a method of cleaning comprising contacting an effective amount of a cleaning or laundry composition comprising a lysozyme polypeptide described herein with an item or surface comprising a soil, stain or biofilm to hydrolyze the soil, stain or biofilm.

Other aspects and embodiments of the present compositions and methods will be apparent from the foregoing description and following examples. Various alternative embodiments beyond those described herein can be employed in practicing the invention without departing from the spirit and scope of the invention. Accordingly, the claims, and not the specific embodiments described herein, define the scope of the invention and as such methods and structures within the scope of the claims and their equivalents are covered thereby.

### EMBODIMENTS

Embodiment 1. An isolated polypeptide or active fragment thereof having lysozyme activity, wherein the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

Embodiment 2. An isolated polynucleotide comprising a nucleotide sequence that encodes a polypeptide having lysozyme activity, wherein the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

Embodiment 3. A recombinant nucleic acid construct comprising an isolated polynucleotide comprising a nucleotide sequence that encodes a polypeptide having lysozyme activity, wherein the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25 operably linked to a promoter sequence capable of controlling expression of the polynucleotide sequence.

Embodiment 4. An isolated host cell comprising a recombinant nucleic acid construct comprising an isolated polynucleotide comprising a nucleotide sequence that encodes a polypeptide having lysozyme activity, wherein the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25operably linked to a promoter sequence capable of controlling expression of the polynucleotide sequence.

Embodiment 5. A method of producing a polypeptide having lysozyme activity, the method comprising:
a) cultivating a host cell comprising a recombinant nucleic acid construct comprising an isolated polynucleotide comprising a nucleotide sequence that encodes a polypeptide having lysozyme activity, wherein the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25 operably linked to a promoter sequence capable of controlling expression of the polynucleotide sequence under conditions conducive to producing the polypeptide, and
b) optionally, recovering the polypeptide having lysozyme activity.

Embodiment 6. A method for preventing, reducing or removing a biofilm comprising contacting the biofilm with a polypeptide having lysozyme activity or a composition comprising a polypeptide having lysozyme activity.

Embodiment 7. The method of Embodiment 6 where the polypeptide having lysozyme activity has an amino acid sequence having at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 11, 12, 13, 14, 15, 16, 17, 18, 19, 20, 21, 22, 23, 24, and 25.

Embodiment 8. The method of Embodiment 6, where the biofilm is on a textile or hard surface.

Embodiment 9. The method of Embodiment 8, where the hard surface is selected from the group consisting of a laundry machine surface, a dish surface, or a dishwasher surface.

Embodiment 10. The method of Embodiment 6, where the composition is a cleaning composition.

Embodiment 11. The method of Embodiments 6-10, where the cleaning composition is a laundry composition.

Embodiment 12. A method for preventing, reducing or removing a biofilm and/or preventing, reducing or removing microbial growth on a textile or hard surface comprising: (i) contacting a textile or surface with a polypeptide having lysozyme activity or a composition comprising a polypeptide having lysozyme activity; and (ii) optionally, rinsing the textile or surface.

Embodiment 13. The method of Embodiment 12, where the textile comprises a biofilm on a surface of the textile.

Embodiment 14. The method of Embodiment 13, where the biofilm is reduced or removed from the textile.

Embodiment 15. The method of any of Embodiments 6-14, where the biofilm or microbial growth is reduced or removed from the article in an amount selected from the groups consisting of at least 5%, 10%, 15%, 20%, 25%, 30%, 35%, 40%, 45%, 50%, 55%, 60%, 65%, 70%, 75%, 80%, 85%, 90%, 95% or greater compared to the amount of the biofilm present on the textile or hard surface prior to contacting the textile or hard surface with the polypeptide having lysozyme activity or a composition comprising a polypeptide having lysozyme activity.

Embodiment 16. The method of any of Embodiments 6-15, where the biofilm is measured using the method of Example 1.

Embodiment 17. The method of any of Embodiments 6-16, where the contacting step comprises the use a polypeptide having lysozyme activity in an amount selected from the group consisting of 0.002 to 10,000 mg of protein, 0.005 to 5000 mg of protein, 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor, or in the amount of at least 0.002 ppm active lysozyme.

Embodiment 18. The method of any of Embodiments 6-17, where the polypeptide having lysozyme activity is a T4 lysozyme.

Embodiment 19. The method of any of Embodiments 6-18, where the contacting step occurs in a wash liquor.

Embodiment 20. The method of any of Embodiments 6-19, where the contacting step takes place for an amount of time selected from the group consisting of about 5 minutes to about 10 days, about 5 minutes to about 400 minutes, between about 5 minutes to about 300 minutes, between about 5 minutes to about 250 minutes, between about 5 minutes to about 200 minutes, between about 5 minutes to about 150 minutes, between about 5 minutes to about 100 minutes, between about 5 minutes to about 50 minutes, between about 5 minutes to about 30 minutes.

Embodiment 21. The method of any of Embodiments 6-20, where the contacting step takes place at a temperature selected from the group consisting of about 10° to 60° C., between 15° to about 55° C., between 20° to about 50° C. and between 20° to about 45° C.

Embodiment 22. The method of any of Embodiments 6-21, where the composition comprising a polypeptide having lysozyme activity further comprises a surfactant.

Embodiment 23. The method of Embodiment 22, where the surfactant is selected from the group consisting of a non-ionic, ampholytic, semi-polar, anionic, cationic, zwitterionic, and combinations and mixtures thereof.

Embodiment 24. The method of any of Embodiments 6-23, where the composition is a detergent composition.

Embodiment 25. The method of any of Embodiments 6-24, where the contacting step further includes contacting the textile or hard surface with one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

Embodiment 26. The method of any of Embodiments 6-25, where the contacting step takes place in a washing machine or a dishwasher.

Embodiment 27. A detergent composition comprising: (i) a polypeptide having lysozyme activity; (ii) a polypeptide having protease activity; (iii) optionally, at least one additional polypeptide, where the at least one additional polypeptide is an enzyme selected from: acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof; and (iv) a surfactant.

Embodiment 28. The composition of Embodiment 27, where the surfactant is selected from the group consisting of a non-ionic, ampholytic, seini-poiar, anionic, cationic, zwitterionic, and combinations and mixtures thereof

Embodiment 29. The composition of Embodiments 27-28, where the composition comprises between about 0.1% to about 60%, about 1% to about 50%. or about 5% to about 40% surfactant by weight of the composition.

Embodiment 30. The composition of Embodiments 27-29, where the polypeptide having lysozyme activity has an amino acid sequence having at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

Embodiment 31. The composition of Embodiments 27-30, where the nuclease is a DNase.

Embodiment 32. The composition of Embodiments 27-31, where the composition further comprises one or more adjunct materials selected from the group consisting of builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents.

Embodiment 33. A method for preventing, reducing or removing microbial growth in a liquid detergent solution comprising including in a liquid detergent solution an effective amount of a lysozyme and a surfactant.

Embodiment 34. The method of Embodiment 33, wherein the lysozyme has an amino acid sequence having at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

Embodiment 35. The method of Embodiments 33 or 34, wherein the liquid detergent solution is a laundry or dish detergent.

Embodiment 36. The method of any of Embodiments 33-35, wherein the liquid detergent solution comprises a lysozyme in an amount selected from the group consisting of to 10,000 mg of protein, 0.005 to 5000 mg of protein, 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor, or in the amount of at least 0.002 ppm active lysozyme.

Embodiment 37. The method of any of Embodiments 33-36, wherein the surfactant is selected from the group consisting of a non-ionic, ampholytic, semi-polar, anionic, cationic, zwitterionic, and combinations and mixtures thereof.

Embodiment 38. The method of any of Embodiments 33-37, wherein the liquid detergent solution comprises further includes one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

Embodiment 39. Use of a lysozyme for preventing, reducing, or removing microbial growth in a liquid detergent.

Embodiment 40. A composition comprising at least 0.002 mg of a polypeptide having lysozyme activity, wherein the polypeptide has an amino acid sequence having at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

Embodiment 41. The composition of Embodiment 40, wherein the composition comprises the polypeptide having lysozyme activity in an amount selected from the group consisting of 0.002 to 10,000 mg of protein, 0.005 to 5000 mg of protein, 0.01 to 5000 mg of protein, 0.05 to 5000 mg of protein, 0.05 to 1300 mg of protein, 0.1 to 1300 mg, of protein, 0.1 to 500 mg of protein, 0.1 to 100 mg of protein.

Embodiment 42. The composition of Embodiments 40-41, wherein the composition is a detergent composition.

Embodiment 43. The composition of Embodiment 42, wherein the detergent composition is a laundry detergent composition or a dishwashing detergent composition.

Embodiment 44. The composition of any of Embodiments 40-43, wherein the composition further comprises one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof

Embodiment 45. A method for preventing, reducing or removing microbial growth in a liquid composition comprising including in the composition an effective amount of a lysozyme, wherein the lysozyme has an amino acid sequence having at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, and 25.

Embodiment 46. The method of Embodiment 45, wherein the liquid composition further comprises a surfactant.

Embodiment 47. The method of any of Embodiments 45 or 46, wherein the liquid composition comprises a lysozyme in an amount selected from the group consisting of 0.002 to mg of protein, 0.005 to 5000 mg of protein, 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor, or in the amount of at least 0.002 ppm active lysozyme.

Embodiment 48. The method of any of Embodiments 46-47, wherein the surfactant is selected from the group consisting of a non-ionic, ampholytic, semi-polar, anionic, cationic, zwitterionic, and combinations and mixtures thereof

Embodiment 49. The method of any of Embodiments 45-48, wherein the liquid detergent solution comprises further includes one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

### EXAMPLES

### Example 1

### Dispersal of Biofilms by Lysozyme

### Methods

A biofilm dispersal assay was adapted from the procedure described by Pitts et al (Pitts, B., Hamilton, M. A., Zelver, N., Stewart, P. S. (2003) A microtiter-plate screening method for biofilm disinfection and removal. Journal of Microbiological Methods 54: 269-276). To produce biofilms in 96-well plates, Pseudomonas fluorescens (ATCC 700830) was grown overnight (18-24 hours) at 28° C., 200-220 rpm with Tryptic Soy Broth (TSB, Teknova T11550). Cultures were diluted to approximately 0.1 OD600 units. These dilutions were used to seed microtiter plates (PVC U bottom 96 well plates, Corning 2797) with 100 μL per well×96 wells. The plates were sealed with breathable film or foil. The seeded microtiter plates were incubated for approximately 48 hours at 26° C. without agitation. The liquid was decanted and the plates were washed 3-5 times with phosphate buffered saline, then allowed to dry.

To simulate the wash treatment, approximately 150 μL of simulated wash solution was added to each well either with or without T4 lysozyme (MCLabs, South San Francisco, CA) added. The simulated wash solution consisted of Tide Original liquid laundry detergent at the approximate recommended laundry dosage (1:1200 dilution in water). The plates were sealed with foil and mixed briefly in a shaker (approximately 300 rpm), then incubated at 26° C. for 35-45 minutes with intermittent shaking to simulate a laundry wash cycle. The simulated wash solutions were decanted and the plates were washed 4-7 times with water. The plates were allowed to dry.

Biofilms were detected with crystal violet as follows. A 0.1% solution of Crystal violet was dispensed into each well, 150 μL per well. The plates were incubated at room temperature for a minimum of 10 minutes. The crystal violet solution was decanted and the plates were rinsed 3-5 times with water. The plates were allowed to dry. Additional stain was removed with a solution of 30% acetic acid, 150 μL per well. The absorbance at 590 nm of this solution was measured on a spectrophotometer.

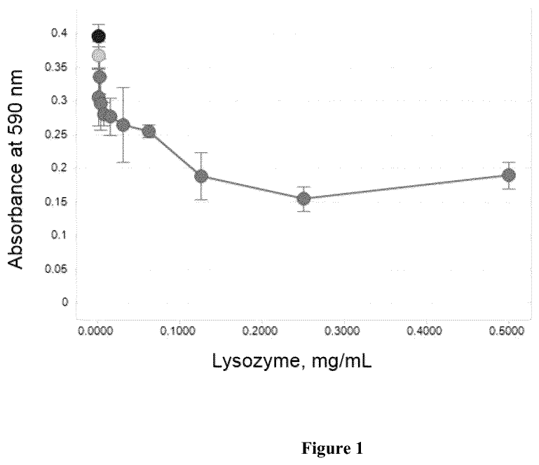

In a single simulated laundry wash cycle, approximately 50% reduction in crystal violet biofilm signal was seen in simulated wash solutions containing T4 lysozyme relative to wash solutions without T4 lysozyme (FIG. 1).

### Example 2

### Identification and Cloning of Lysozymes

Lysozyme genes from Glycoside hydrolase family 19, 22, 24 and 25 were collected from the NCBI database, JGI database and internal sequencing data based on PFAM prediction. Redundant genes were removed and phylogenetic analysis of lysozyme in each GH family was conducted. Representative genes covering a wide sequence diversity were selected for cloning and expression. Generally, fungus-originated lysozyme genes were cloned into the pGX256 vector and transformed into Trichoderma reesei for expression, while bacterium and phage-originated ones cloned into the p2JM or p3JM vector and transformed into Bacillus subtilis for expression. The fermentation crude was used for preliminary activity test with Micrococcus lysodeikticus as substrate. Then promising molecules were subjected to large-scale fermentation and further purification using standard protein purification techniques.

### Example 3

### Reduction of Biofilms by Lysozymes

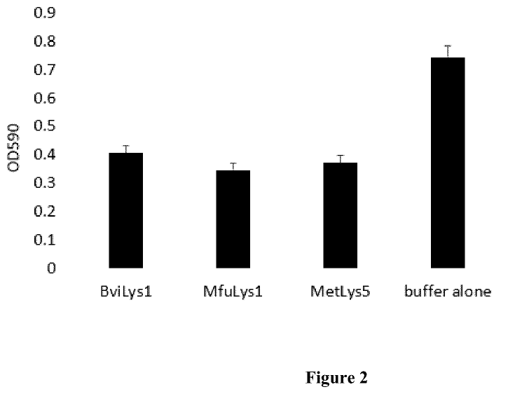

A biofilm dispersal assay was adapted from the procedure described by Pitts et al (Pitts, B., Hamilton, M. A., Zelver, N., Stewart, P. S. (2003) A microtiter-plate screening method for biofilm disinfection and removal. Journal of Microbiological Methods 54: 269-276), briefly as follows. Pseudomonas fluorescens (ATCC strain 700830) biofilm was formed on 96-well round bottom plates (Corning 2797). Briefly, seed cultures from an LB growth plate were inoculated in fresh TSB media followed by OD600 adjustment to 0.1-0.2. Then the cell suspension was transferred to a microtiter plate and the plate was incubated in an oxygen chamber statically for 48 h at 28° C. After washing 5 times with 1×PBS and air-drying, the biofilm buildup in plates was treated with enzyme solution at 270 PPM, prepared in 50 mM HEPES buffer pH 8.0 (buffer alone used as negative control). For each sample, eight replicates were performed. Plates were incubated in an iEMS incubator at 26° C. with shaking at 400 rpm for 2 hours. Then the treatment solutions were decanted, and the plate was washed 5 times with Milli-Q water and air dried. After treatment, biofilm was stained by crystal violet solution (0.1%). After 5 min, the excess crystal violet was removed and the plate was washed 5 times and air dried. Finally, the biofilm bound crystal violet was dissolved in 30% acetic acid solution. Biofilm was monitored in terms of OD590 nm using a spectrophotometer. As shown in FIG. 2, the biofilms were substantially reduced relative to those in an untreated control.

### Example 4

### Reduction of Biofilms by Lysozymes in Laundry Detergent Solutions

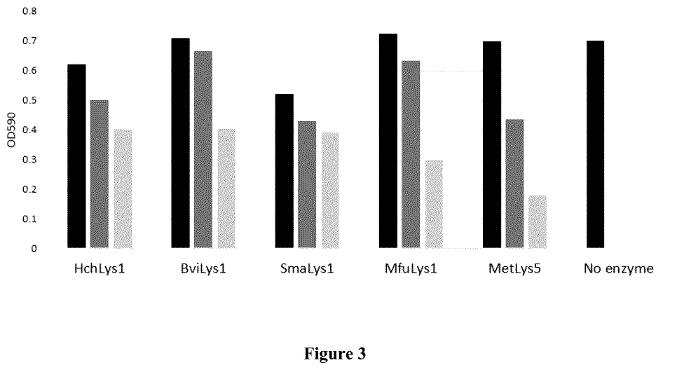

A biofilm dispersal assay was adapted from the procedure described by Pitts et al (Pitts, B., Hamilton, M. A., Zelver, N., Stewart, P. S. (2003) A microtiter-plate screening method for biofilm disinfection and removal. Journal of Microbiological Methods 54: 269-276), briefly as follows. Pseudomonas fluorescens (ATCC strain 700830) biofilm was formed on 96-well round bottom plates (Corning 2797). Briefly, seed cultures from an LB growth plate were inoculated in fresh TSB media followed by OD600 adjustment to 0.1-0.2. Then the cell suspension was transferred to a microtiter plate and the plate was incubated in an oxygen chamber statically for 48 h at 28° C. After washing 5 times with 1X PBS and air-drying, the biofilm buildup in plates was treated with enzyme solution at 10 PPM, 50 PPM, or 250 PPM, prepared in a laundry wash solution of Tide Original liquid detergent diluted 1:1200 in water. For each sample, eight replicates were performed. Plates were incubated in an iEMS incubator at 26° C. with shaking at 400 rpm for 400 minutes. Then the treatment solutions were decanted, and the plate was washed 5 times with Milli-Q water and air dried. After treatment, biofilm was stained by crystal violet solution (0.1%). After 5 min, the excess crystal violet was removed and the plate was washed 5 times and air dried. Finally, the biofilm bound crystal violet was dissolved in 30% acetic acid solution. Biofilm was monitored in terms of OD590 nm using a spectrophotometer. As shown in FIG. 3, substantial reduction in biofilm signal was seen relative to the no-enzyme controls.

### Example 5

### Antimicrobial Activity Against Laundry Malodor Species Micrococcus luteus and Moraxella osloensis

Two microorganism species relevant to laundry hygiene and malodor were ordered from the American Type Culture Collection (ATCC): Moraxella osloensis (ATCC 19976) and Micrococcus luteus (ATCC 4698). Each organism was cultured overnight in BHI media at a permissive temperature. The overnight cultures' turbidity was measured at 600 nm, and a volume of cells corresponding to 0.5 OD₆₀₀ was withdrawn and pelleted by centrifugation. The cells were washed twice with 1×phosphate buffered saline solution (PBS) to remove traces of the growth medium and resuspended in 1 mL of PBS after the second wash. The cell suspensions were kept on ice to prevent growth during the remainder of the preparation phase.

When possible, CFU enumeration from the cell suspensions was performed to accurately gauge the number of cells in each well of the simulated wash plate. Briefly, the cell suspensions were diluted by a factor of 10⁴ to 10⁶ in PBS and 100 μL of each dilution plated until a countable number of colonies arose after overnight incubation. From this enumeration process, cell counts in the simulated wash were approximately 7×10⁵ CFU/well of Micrococcus and 3×10⁵ CFU/well of Moraxella.

Lysozymes were obtained commercially (MCLabs, South San Francisco, CA) or they were produced by standard molecular biology techniques to enable expression in bacterial or fungal host cells. Stock solutions of lysozymes were diluted into a sterilized solution of phosphate buffered saline, pH 7.2, (PBS) to obtain final lysozyme concentrations of 0, 1.56, 3.13, 6.25, 12.5 or 25 PPM in the final simulated wash solution.

In sterile 96-well microtiter plates, 2 μL cell suspension of each microorganism was treated with 125 μL of the PBS solution with lysozyme added, as well as no-lysozyme negative controls. The plates were agitated at medium speed for 40 minutes at room temperature to simulate a washing machine cycle. Following the wash, the cells in the wash plate were allowed to recover overnight in liquid growth media to assess survival after treatment.

Twenty microliters of each well of the simulated wash plate were transferred to new plates containing 180 μL of appropriate liquid growth medium (YPD for the yeast, BHI for the bacteria). The plate was incubated at a permissive temperature without agitation overnight. The next day, the plates were agitated to homogenize the well contents and each well's OD₆₀₀ was read on a plate reader. Survival curves plotting turbidity against lysozyme concentration were generated.

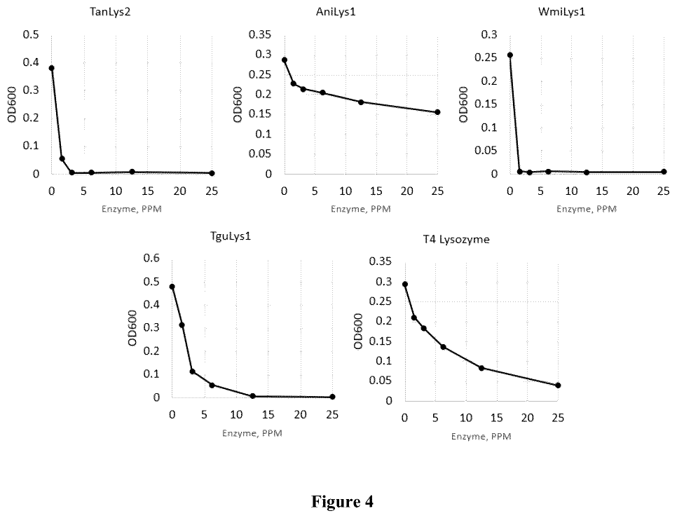

The results of liquid culture outgrowth tests of Micrococcus luteus following the small-scale laundry simulation tests for seven lysozymes at varied concentrations, as well as a no-enzyme control are provided in FIG. 4. Addition of lysozyme led to reduction of Micrococcus luteus bacterial growth at concentrations above 1.56 PPM for all lysozyme variants.

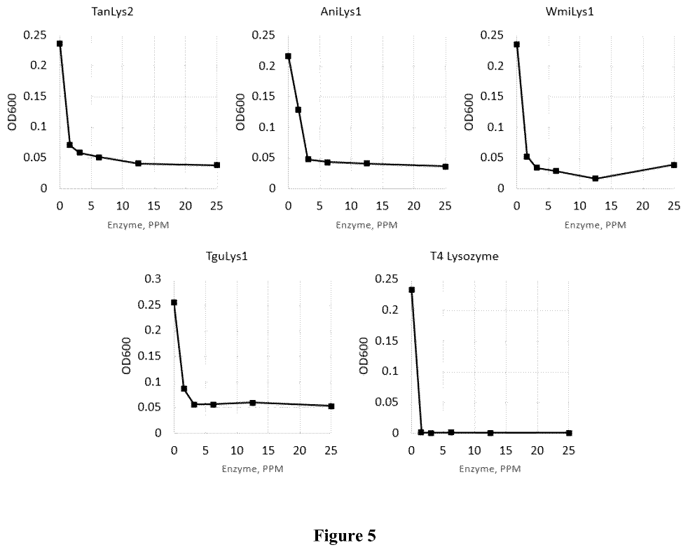

The results of liquid culture outgrowth tests of Moraxella osloensis following the small-scale laundry simulation tests for seven lysozymes at varied concentrations, as well as a no-enzyme control, are provided in FIG. 5. Addition of lysozyme led to reduction of Moraxella osloensis bacterial growth at concentrations above 1.56 PPM for all lysozyme variants.

### Example 6

### Antimicrobial Activity in Mid-Scale Laundry Applications Tests

The antimicrobial activity of two experimental lysozymes was investigated against Micrococcus luteus in a midscale laundry assay at 25° C. for 30 minutes. Thawed frozen cells of M luteus were grown on brain heart infused agar media at 26° C. for 18 hours. A single bacterial colony was used to inoculate 3mL of brain heart infused liquid media. The liquid culture was incubated at 26° C. for 18 hours with constant mixing (150 rpm). A subculture was prepared by transferring 300 μL of the previous culture to 3mL of fresh BHI liquid media and incubated at 26° C. for 18 hours with constant mixing (150 rpm). The subculture step was repeated a second time to give a starting optical density (A600) equivalent approximately 10 9 viable cells per mL. At the start of the midscale test the liquid culture was diluted to an optical density of 0.04 (OD600) against 100 mL of sterile water. A commercially available household liquid detergent (Tide Original Liquid) was purchased and prepared to a final concentration of 0.8 mg detergent per mL of deionized water. A solution of dissolved calcium and magnesium at a molar ratio of 3:1 was prepared to approximate North American water hardness levels around 150 PPM. The experimental lysozymes were purified and diluted to a concentration of 20 μg of enzyme per mL of deionized water. The enzymes were assayed at 2 PPM in the presence of detergent and water hardness and also in detergent alone.

The midscale laundry assay was executed using the Launder-Ometer instrument (ATLAS). The Launder-Ometer was filled with water to the recommended water level and pre-equilibrated to 25° C. for 30 minutes prior to the start of the experiment. Launder-Ometer pots were filled to a volume of 200 mL with each of the experimental conditions. Pots were capped and locked in place on the instrument and the laundry assay was initiated. At the end of 30 minutes, 5 mL of wash liquor was removed from each condition for analysis.

Colony forming units were determined by inoculating 1004, of diluted wash liquor on brain heart infused agar media plates. The plates were incubated at 26° C. for 72 hours. Table 1 shows the results from colony counting.

TABLE 1
Results

| | Bacterial count based on |
|---|---|
| Condition | colonies on agar plate |
| Tide Original liquid laundry detergent | 7.2 × 10⁴ cfu/mL |
| Tide Original + 2 PPM TanLys2 + water | No colonies |
| hardness | |
| Tide Original + 2 PPM WmiLys1 + water | No colonies |
| hardness | |

### Example 7

### Reduction of Biofilms in Laundry Detergent Solutions

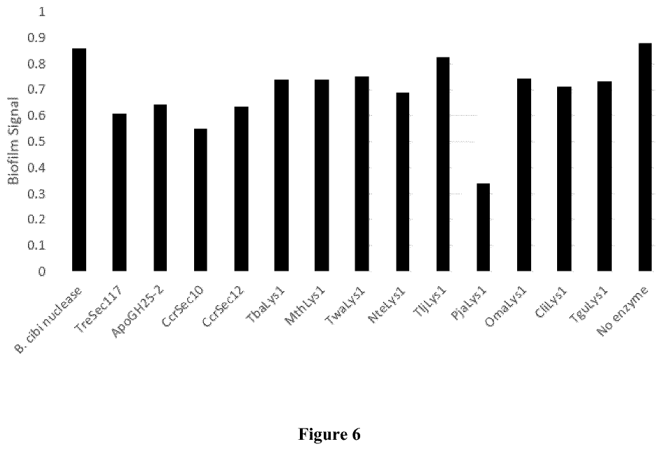

Pseudomonas fluorescens (ATCC strain 700830) biofilm was formed on 96-well round bottom plate (Corning 2797). Briefly, seeds from LB plate was inoculated in fresh Tryptic Soy Broth (TSB, Teknova T11550) followed by OD600 adjustment to 0.1-0.2. Then the cell suspension was transferred to a microtiter plate and the plate was incubated in oxygen chamber statically for 48 h at 28 degrees C. After decanting and washing the plate 5 times with 1X PBS and air-drying, the biofilm buildup in plates was treated with enzyme solution at the concentration of 50 ppm, prepared in 1:1200 dilution of Tide Original liquid detergent (Tide solution alone used as negative control). For each sample, eight replicates were performed. Plates were incubated in iEMS incubator at 26 degrees C. with shaking at 400 rpm for 400 min. Then treatment solutions were decanted, and the plate was washed 5 times with Milli-Q water and air dried. After treatment, biofilm was stained by crystal violet solution (0.1%). After 5 min, the excess crystal violet was removed and the plate was washed 5 times and air dried. Finally, the biofilm bound crystal violet was dissolved in 30% acetic acid solution. Biofilm was monitored in terms of OD590 nm using a spectrophotometer. The average biofilm signal is plotted in FIG. 6 with comparisons to a no enzyme control and also to Bacillus cibi nuclease (e.g. WO2018/011277A1).

### Example 8

### Lytic Activity Toward Micrococcus lysodeikticus

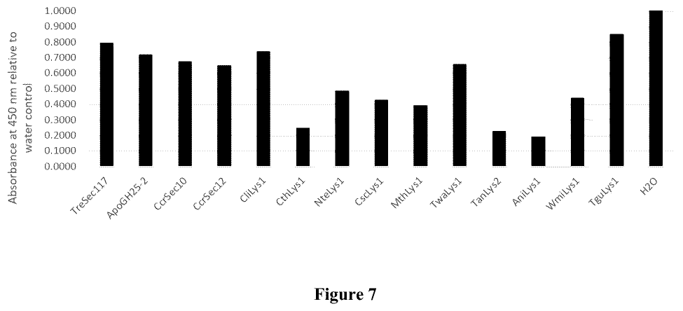

Lysozymes were tested for their lytic activity towards Micrococcus lysodeikticus cells using the Lysozyme Detection Kit (Sigma, LY0100). Substrate suspension was prepared as 0.05% w/v Micrococcus lysodeikticus cell in lysozyme reaction buffer (66 mM potassium phosphate, pH 6.24). The reaction was initiated by transferring 10 μL of enzyme dilution (or water, used as negative control) into 190 μL of substrate suspension. The final concentration of enzyme in the assay was 50 PPM. The reaction was incubated at 25 degrees C. shaking at 400 rpm in an iEMS incubator. After 2 hours, the absorbance of the reaction solution was determined at 450 nm in a spectrophotometer. FIG. 7 shows the absorbance of each sample at 450 nm divided by the absorbance of the no-enzyme (water only) sample.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

Sequence Listing

SEQ ID NO: 1>, CscLys1
AIGSQCFVNNVPGVCLTTTTCSSGGGTSTAGYCPNDPANVRCCTKACGSGGRCRTVNSCVSGNILSGLCPGDSNVKCCLPASAGCSSGP
AVNTATLNLVKASEGWRPNIYTDPAGLATVGYGHLCQKAKCAEVPYSIPLSVADGTRLLQSDMKTAQNCLAKAVSVKLNANQYGAL
VSWTFNVGCGAMRGSALVERLNKGQTPNTVASEELPRWNKGGGKVLPGLVTRRAKEVTLFKTATTAKGIPC
SEQ ID NO: 2>, OmaLys2
YTITGDSVNCRSGPGTSYAVKKTYAKGHDVSISCQAPGTSVDGDSLWDKTSDNCFVADYYVKTGTSGYVAAHCSSGSGGSSSCAAPAS
NTATVNLIAEFEGFVDHVYTDATGHPTIGYGHLCSNSACSGIGYPIPLSQANGKKLLAKDMGVAEKCITAMTNDKVTLNLNQYGALVS
WAFNEGCGAAKSSTLIKRLNNGESPKTVLPQELPKWVYGNGEVLPGLVRRRKAEVALSEEATTAKALPVKC
SEQ ID NO: 3> T4 lysozyme
MNIFEMLRIDEGLRLKIYKDTEGYYTIGIGHLLTKSPSLNAAKSELDKAIGRNCNGVITKDEAEKLFNQDVDAAVRGILRNAKLKPVYDS
LDAVRRCALINMVFQMGETGVAGFTNSLRMLQQKRWDEAAVNLAKSRWYNQTPNRAKRVITTFRTGTWDAYKNL
SEQ ID NO: 4>, CthLys1
YAITGDNVNCRSGPGTSYAVKKVYKKGTDVKISCQTTGTNINGNNLWDKTSDGCYVSDYYVKTGSNGYVTSKCSSSGGSTCAAPKSN
QATVDLIAEFEGFRANIYTDAAGYATVGYGHKCQKAKCAEVKYKIPLSKADGKKLLADDMRSFEVCITNMLNSKAKLNYNQFGALVS
WSFNVGCGAAKSSTLIKRLNNGENVNKVLSEELPKWNKAGGKVLQGLVRRRAAEVALAKKSGSSQALPVKC
SEQ ID NO: 5>, TanLys2
YLITGDTVNCRSGPGTSYAVKKTYTKGQDVKISCQQAGTSVDGNNIWDKTQDGCFVADRYVKTGVAGYVTEKCGVKTCAAPAVNQA
TVDLIAEFEGFRASVYIDATGHPTVGYGHLCTQAKCAEVKYKIPLSQADGKKLLADDIKNYEKCVTSMANSNAKLNANQYGAVVSFTF
NLGCGAAQGSQMMKRLNAGENPNTVIANEFPKWVYGNGKVLPGLVRRRDAEVALAKKATSTAAIPPKC
SEQ ID NO: 6>, AniLys1
ATHLLPRAVGDSCTAPEGKGTCQNTSNCAGISYPTGLCPNDPTNVQCCVKISCSTSSGSGYCRSVSNNGCSGGTFISGACPGSSDIRCCV
KSSGGGTGGGDGHIDQYSINHLKSLEGVRRYFYYDSVGKKTIGVGHNCDAHPGTCDNLVEPLTDAEIDRLLRMDLQIFETCVCKMANA
SLMNKYQFAALVSFAFNSGCGGASRYFADEMKSKSFSKICTDLPTTNTLNGLLTSRRTKEKELCQKATTVKSGC
SEQ ID NO: 7>, WmiLys1
YPTKIILNCRSSPSTSSSIIRTYPKGYDIKISCQTTGTKVETSNVWDKTQHGCYVSDYYVSTGHAGIFLTTCGSTGGGSTCGPPNINAATID
LIKSFEGFVASPSPDPVGLPTVGYGHLCKTKGCSEVPYPFPLTHATAAMLLQDDAREFKACVSNAIVNSVTLNDNQYGALVSWAFNVG
CGNVRSSSLVRRLNRGENKNVVVSQELIKWNKAGSPLRVLPGLTRRRNAEIALFKTPSSVQAHPPKC
SEQ ID NO: 8>, HchLys1
MCIKSSVGLHGTNSKTDVKVIQAALNLYTSGSFLLESKLTVDGQIGPKTIQAITLLQKSSVQISKPDGKVDPKGKTLKTLKQGVTKGLSE
YALAAIMAHGKSSVINKYFPLLQNNLSRYQINSPLRIAHFLAQVGHESLSFRYTEELASGANYEGNLALGNTQSGDGVRFKGRGLIQLT
GRSNYSEYAEYSRIDLMKKGNEVLVALTPAYALDVSLWFWNKRRLNTKADKDDLRGVTYRVNGGYTGLQDRRDYLDRAKFFLLP
SEQ ID NO: 9>, BviLys1
ALNGPCTVGSTPGVCITTTTCSSGGGTTHSGYCPNDADDVKCCTKTCGSGGTCKFSSSCSGTTQSGLCPGPTDFKCCLPSSGSSCTVGPS
VNSATVTLIKNSEGFVSKPAPDPIGLPTVGYGHLCQKSNCSEVPYSFPLTTTTATQLLQSDLKTYQNCVAQMIKVRLNANQYGALVSFTF
NMGCSAAKGSTLVARLNAGENPNTVAAQELPKWVNAGGQQLPGLVTRRKNEVALFQTATSTGALPC
SEQ ID NO: 10>, MfuLys1
APSVSEERACAPPDVNAATVNLIKQFEGFVASPQNDPVGLPTVGYGHLCKSKNCAEVPFKFPLTQDDAAKLLQTDLKTFENCVSNDLKP
TVKLNDNQYGALTSWAFNVGCGNVGSSDLVKRMNAGEDPTAVAQSELPQWNKGGGKVLPGLTRRRAAEVALFKTPSSVIAHPAC
SEQ ID NO: 11>, MetLys5
MDKNIKEIQTLLIQGGFSVGSSGADGLYGNDTRNALIACINKANSGEKKLKLTLEQLNKIFPAGASSGRNAKFIDPLNELFEARQINTVNR
IAGFLSQVGVESEEFLYTRELGNAAYFNKYDIQYAPQKAKDLGNTQPGDGAKFKGRGLIQVTGRANYTACGKALGLDLVNRPELLEQP
KYAVDSAGWYWGLRNINAACDANDIVAITKKVNGGTMHLDRRTAYYNKAKQVLS
SEQ ID NO: 12>, SmaLys1
TVQGFDISHYQSSVNFAGAYSSGARFVIIKATEGTTYTDPKFSSHYTGATSAGLIRGGYHFAHPDSSTGAAQADYFLAHGGGWSNDGIT
LPGMIDLESVSGKATCFGLSTSAMVSWIKSFSDRYHTKTGRYPMIYTNYSWWNQCTGNSKTFATTNPLVLARWSSTIGTLPGGWSVHTI
WQNADTYTYGGDSDVFNGSLDRLKALAKGSG
SEQ ID NO: 13>
TreSec117
TVPGFDISHYQATVDFAKAYADGARFVIIKATEGTTYTDPSFSDHYTKATNAGFIRGGYHFAQPASSSGAAQANYFLKHGGGWSADGI
TLPGMLDLEYAPSGDSCYGLSASAMVSWINDFVNTYHAATTQYPLIYTSTSWWQLCTGNNGSFGSKSPLVIARYASSVGALPNGWSV
YTIWQNSDASPWGGDNDIFNGNLAQLQKIARGS
SEQ ID NO: 14>
ApoGH25-2
APLGKRGSIPGIDVSHYQGSINWSTVKANGVQFVYIKATEGTTYQDPQFSNNYVGATNVGIYRGGYHFARPNLSSGAAQANYFIAHGG
GWTSDGRTLPGALDIEYNDDGAECYGLSAASMVSWIKDFSNTYHSKLGVAAFASTNPLWVARYSSSVGTLPAGWSYETFWQHADSG
SNPGDQDIFNGDAAGLKRLALG
SEQ ID NO: 15>
CcrSec10
TVQGFDISHYQTNVNFAAAYNSGARFVMIKATESTTYIDPSFNSHYTSATSAGFIRGGYHFAVPSDSSGATQANYFLAHGGGWSGDGIT
LPGMLDIEYNPYGATCYGLSASQMVSWIADFVNTYKSKTGRPPMIYTTADWWNTCTGNSNSFTECPLVLARYSSSVGTIPGGWPYQSF
WQNSDSYAYGGDSDIWNGSLDNLKKFASG
SEQ ID NO: 16>
CcrSec12
LPSQPEAGATTVQGFDISNHQKSANFEAAKKDGAQFVIIKATEGTTFKDPVFNSHYTGATKAGLIRGGYHFARPDTSTGSAQAKYFLKN
GGGWSNDNRTLPGMLDIEYNPYGATCYGLSHSQMVAWIHDFVDEYHHATSRWPMIYTTADWWNRCTGNAKGFGDKCPLVLAAYRS
TPPTTIPGDWRTWTIWQNSDKYEHGGDSDKFNGPMKQLRKLASG
SEQ ID NO: 17>
TbaLys 1
AVQGFDISSYQGSIDWSGAYSSGARFVIIKATEGTDYIDSGFNSHYTGATNAGFIRGGYHFAHPDSSSGADQAKYFLAHGGGWSNDGIT
LPGMLDIEYNPSGNECYGLSASAMVSWITDFVNTYHSATGRYPMIYSTDDWWSTCTGNSDAFSSNCPLVLARYGSSPGTIPGGWPYQTI
WQNADSYTYGGDSDVFNGSLDNLKKLASG
SEQ ID NO: 18>
MthLys1
AVQGFDISHYQPSVDFAAAYKSGARFVIIKATEGTSYIDPKFSSHYTGATKAGFIRGAYHFAHPGQSSGEAQADYFLAHGGGWTSDGIT
LPGMLDLEAYNAGQCWGLSTSAMVAWIKAFSDRYHSRTGVYPLLYTNPSWWKACTGNSNAFVNTNPLVLARYASSPGEIPGGWPYQ
TIWQNSDSYAYGGDSDIFNGDLDGLKRLAKGP
SEQ ID NO: 19>
TwaLys1
APAPEKRASGVQGCDISNYQPNFSYASAADAGAKFVIIKATEGTSYTSPSFSRQYSGATDAGFLRGAYHFAHPDSSSGAAQANFFLANG
GGWSGDGRTLPGMVDLEYNPAKNSNSCYGLSQSAMVSWIRSFTSTYYSKTGRYPMIYTTNDWWRTCTGNSDAFSSTSPLVLARYSSSG
PGTIPGGWPYQTIWQNSDSFAAGGDSDIFNGSLDGLKRLASG
SEQ ID NO: 20>
NteLys1
TVQGFDISHYQGSVNFARAYSSGARFVIIKATEGTNYIDPKFSSHYTGATSAGLIRGGYHFAHPDSSSGAAQADYFLAHGGGWSKDGITL
PGMIDLESVSGKATCYGLSTSAMVSWIKSFSDRYHSKTGRYPMIYTNYSWWSKCTGNSKSFATTNPLVLARWASSVGTIPGGWSYQTI
WQNADTYTYGGDSDIFNGSLDRLKALAKGS
SEQ ID NO: 21>
TljLys1
SPILEKRANPKGIDISHFQGTVNFNTVKANGISFVYIKATEGTTFTDPDFSSHYTGATNAGLIRGAYHFAHPDVSSGATQAKFFLAHGGG
WSSDGITLPGALDIEYNPSGAECYGLSASAMVSWIKDFSNTYHSSTGVYPFIYTTTDWWKTCTGNSAAFASTNPLWIARYASSVGTLPA
GWSYHTFWQYADSGPNPGDQDEFNGSMQGLKNLALG
SEQ ID NO: 22> PjaLys1
YPVDADDLHCRSGPGTNYGIVKSYKRGTELTITCQAAGTNVNGDELWDKTSDGCYVTDYYVKTGTSGYVTKHCDSGSTGGGGGSSGH
GANEATLKLIGQLEGWRPNFYYINGHKTIGYGHDCVEKGCSGINPPLTQQQGLDLLKKDIVGFEDCVCNLPNAKELNANEYGALVSFA
YNSGCGGVSRYWHSAMEQKNFKGICEALPHTNTLGGELNNRRKQEGDFCSTPTSEKAGC
SEQ ID NO: 23> OmaLys1
APSTAIEARNASPINAGAISLIESLEGFRADFYYINGHETIGYGHDCVESGGCGSLHPPISQAEGTALFKKDIAEYESCVCAMANAKDLNA
NQYGALVSFAYNSGCGGVQSWWHGAMAKKNFKGICEALPTTNTLGGELSSRRKKEGAFCSKATTAKSGCA
SEQ ID NO: 24> CliLys1
FDISHYQSSVNYAGAYAAGARFVIIKATEGTTYTDPSFSTHYTGATNAGLIRGGYHFAHPGETTGAAQADYFITHGGGWSGDGITLPGM
LDLESEGGATCWGLSTSAMVAWIKAFSDRYHSQTGRYPMLYTNPSWWTSCTGNSNAFVSTNPLVLARYASAPGTIPGGWPYQTIWQN
SDSYAYGGDSDIFNGSLDNLRKLATG
SEQ ID NO: 25>
TguLys1
APALEERASYVQGFDISHYQGTVNFKSAYSSGARFVIIKATEGTSVVDAGFSSHYSGATSAGLIRGGYHFAHPDESSGATQANFFLAHGG
GWSNDGITLPGMLDIEYNPSGATCYGLSQSSMVSWIKDFADTYHSKTSRYPLIYTTNDWWTTCTGDSTAFYTTSPLVLARYGSSPGTIP
GGWPYETIWQNADSYTYGGDSDKFNGAESSLKKLATG

1. An isolated polypeptide or active fragment thereof having lysozyme activity, wherein the polypeptide has at least 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1, 2, 4, 6, 7, 8, 9, 10, 11, 12, 13, 14, 15, 16, 17, 18, 19, 20, 21, 22, 23, 24, and 25.

2. (canceled)

3. (canceled)

4. An isolated host cell comprising a recombinant nucleic acid construct comprising an isolated polynucleotide comprising a nucleotide sequence that encodes the polypeptide of claim 1 operably linked to a promoter sequence capable of controlling expression of the polynucleotide sequence.

5. (canceled)

6. A method for preventing, reducing or removing a biofilm comprising contacting the biofilm with a polypeptide of claim 1 or a composition comprising the polypeptide of claim 1.

7. (canceled)

8. The method of claim 6, wherein the biofilm is on a textile or hard surface.

9. (canceled)

10. The method of claim 6, wherein the composition is a cleaning composition.

11. (canceled)

12. A method for preventing, reducing or removing a biofilm and/or preventing, reducing or removing microbial growth on a textile or hard surface comprising: (i) contacting a textile or a hard surface with the polypeptide of claim 1; and (ii) optionally, rinsing the textile or surface.

13. The method of claim 12, wherein the hard surface is selected from the group consisting of a laundry machine surface, a dish surface, a medical instrument or a dishwasher surface.

14. (canceled)

15. The method of claim 12, wherein the biofilm or microbial growth is reduced or removed from the article in an amount selected from the groups consisting of at least 5%, 10%, 15%, 20%, 25%, 30%, 35%, 40%, 45%, 50%, 55%, 60%, 65%, 70%, 75%, 80%, 85%, 90%, 95% or greater compared to the amount of the biofilm present on the textile or hard surface prior to contacting the textile or hard surface with the polypeptide or a composition comprising said polypeptide.

16. (canceled)

17. (canceled)

18. (canceled)

19. (canceled)

20. (canceled)

21. (canceled)

22. (canceled)

23. (canceled)

24. (canceled)

25. The method of claim 6, wherein wherc the contacting step further includes contacting the textile or hard surface with one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hem icellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

26. The method of claim 6, wherein whcrc the contacting step takes place in a washing machine or a dishwasher.

27. A detergent composition comprising: (i) the polypeptide of claim 1; (ii) a polypeptide having protease activity; (iii) optionally, at least one additional polypeptide, where the at least one additional polypeptide is an enzyme selected from: acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof; and (iv) a surfactant.

28. (canceled)

29. (canceled)

30. (canceled)

31. The composition of claim 27, wherein the nuclease is a DNase.

32. The composition of claim 27, wherein the composition further comprises one or more adjunct materials selected from the group consisting of builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents.

33. A method for preventing, reducing or removing microbial growth in a liquid detergent solution comprising including in a liquid detergent solution an effective amount of the polypeptide of claim 1 and a surfactant.

34. (canceled)

35. The method of claim 33, wherein the liquid detergent solution is a laundry or dish detergent.

36. The method of claim 33, wherein the liquid detergent solution comprises a lysozyme in an amount selected from the group consisting of 0.002 to 10,000 mg of protein, 0005 to 5000 mg of protein, 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor, or in the amount of at least 0.002 ppm active lysozyme.

37. (canceled)

38. The method of claim 33, wherein the liquid detergent solution comprises further includes one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

39. Use of a lysozyme for preventing, reducing, or removing microbial growth in a liquid detergent.

40. (canceled)

41. (canceled)

42. (canceled)

43. (canceled)

44. (canceled)

45. (canceled)

46. (canceled)

47. (canceled)

48. (canceled)

49. (canceled)


SEQUENCE LISTING (25 sequences)

SEQ ID NO: 1 - protein, 246 aa, Coprinopsis sclerotiger
AIGSQCFVNNVPGVCLTTTTCSSGGGTSTAGYCPNDPANVRCCTKACGSGGRCRTVNSCVSGNILSGLCPGDSNVKCCLPASAGCSSGPAVNTATLNLVKASEGWRPNIYTDPAGLATVGYGHLCQKAKCAEVPYSIPLSVADGTRLLQSDMKTAQNCLAKAVSVKLNANQYGALVSWTFNVGCGAMRGSALVERLNKGQTPNTVASEELPRWNKGGGKVLPGLVTRRAKEVTLFKTATTAKGIPC

SEQ ID NO: 2 - protein, 247 aa, Oidiodendron maius
YTITGDSVNCRSGPGTSYAVKKTYAKGHDVSISCQAPGTSVDGDSLWDKTSDNCFVADYYVKTGTSGYVAAHCSSGSGGSSSCAAPASNTATVNLIAEFEGFVDHVYTDATGHPTIGYGHLCSNSACSGIGYPIPLSQANGKKLLAKDMGVAEKCITAMTNDKVTLNLNQYGALVSWAFNEGCGAAKSSTLIKRLNNGESPKTVLPQELPKWVYGNGEVLPGLVRRRKAEVALSEEATTAKALPVKC

SEQ ID NO: 3 - protein, 164 aa, Enterobacteria phage T4
MNIFEMLRIDEGLRLKIYKDTEGYYTIGIGHLLTKSPSLNAAKSELDKAIGRNCNGVITKDEAEKLFNQDVDAAVRGILRNAKLKPVYDSLDAVRRCALINMVFQMGETGVAGFTNSLRMLQQKRWDEAAVNLAKSRWYNQTPNRAKRVITTFRTGTWDAYKNL

SEQ ID NO: 4 - protein, 245 aa, Chaetomium  thermophilum
YAITGDNVNCRSGPGTSYAVKKVYKKGTDVKISCQTTGTNINGNNLWDKTSDGCYVSDYYVKTGSNGYVTSKCSSSGGSTCAAPKSNQATVDLIAEFEGFRANIYTDAAGYATVGYGHKCQKAKCAEVKYKIPLSKADGKKLLADDMRSFEVCITNMLNSKAKLNYNQFGALVSWSFNVGCGAAKSSTLIKRLNNGENVNKVLSEELPKWNKAGGKVLQGLVRRRAAEVALAKKSGSSQALPVKC

SEQ ID NO: 5 - protein, 242 aa, Thielavia antarctica
YLITGDTVNCRSGPGTSYAVKKTYTKGQDVKISCQQAGTSVDGNNIWDKTQDGCFVADRYVKTGVAGYVTEKCGVKTCAAPAVNQATVDLIAEFEGFRASVYIDATGHPTVGYGHLCTQAKCAEVKYKIPLSQADGKKLLADDIKNYEKCVTSMANSNAKLNANQYGAVVSFTFNLGCGAAQGSQMMKRLNAGENPNTVIANEFPKWVYGNGKVLPGLVRRRDAEVALAKKATSTAAIPPKC

SEQ ID NO: 6 - protein, 252 aa, Ascodesmis nigricans
ATHLLPRAVGDSCTAPEGKGTCQNTSNCAGISYPTGLCPNDPTNVQCCVKISCSTSSGSGYCRSVSNNGCSGGTFISGACPGSSDIRCCVKSSGGGTGGGDGHIDQYSINHLKSLEGVRRYFYYDSVGKKTIGVGHNCDAHPGTCDNLVEPLTDAEIDRLLRMDLQIFETCVCKMANASLMNKYQFAALVSFAFNSGCGGASRYFADEMKSKSFSKICTDLPTTNTLNGLLTSRRTKEKELCQKATTVKSGC

SEQ ID NO: 7 - protein, 247 aa, Wilcoxina mikolae
YPTKIILNCRSSPSTSSSIIRTYPKGYDIKISCQTTGTKVETSNVWDKTQHGCYVSDYYVSTGHAGIFLTTCGSTGGGSTCGPPNINAATIDLIKSFEGFVASPSPDPVGLPTVGYGHLCKTKGCSEVPYPFPLTHATAAMLLQDDAREFKACVSNAIVNSVTLNDNQYGALVSWAFNVGCGNVRSSSLVRRLNRGENKNVVVSQELIKWNKAGSPLRVLPGLTRRRNAEIALFKTPSSVQAHPPKC

SEQ ID NO: 8 - protein, 263 aa, Hahella chejuensis
MCIKSSVGLHGTNSKTDVKVIQAALNLYTSGSFLLESKLTVDGQIGPKTIQAITLLQKSSVQISKPDGKVDPKGKTLKTLKQGVTKGLSEYALAAIMAHGKSSVINKYFPLLQNNLSRYQINSPLRIAHFLAQVGHESLSFRYTEELASGANYEGNLALGNTQSGDGVRFKGRGLIQLTGRSNYSEYAEYSRIDLMKKGNEVLVALTPAYALDVSLWFWNKRRLNTKADKDDLRGVTYRVNGGYTGLQDRRDYLDRAKFFLLP

SEQ ID NO: 9 - protein, 246 aa, Bolbitius vitellinus
ALNGPCTVGSTPGVCITTTTCSSGGGTTHSGYCPNDADDVKCCTKTCGSGGTCKFSSSCSGTTQSGLCPGPTDFKCCLPSSGSSCTVGPSVNSATVTLIKNSEGFVSKPAPDPIGLPTVGYGHLCQKSNCSEVPYSFPLTTTTATQLLQSDLKTYQNCVAQMIKVRLNANQYGALVSFTFNMGCSAAKGSTLVARLNAGENPNTVAAQELPKWVNAGGQQLPGLVTRRKNEVALFQTATSTGALPC

SEQ ID NO: 10 - protein, 173 aa, Macrolepiota fuliginosa
APSVSEERACAPPDVNAATVNLIKQFEGFVASPQNDPVGLPTVGYGHLCKSKNCAEVPFKFPLTQDDAAKLLQTDLKTFENCVSNDLKPTVKLNDNQYGALTSWAFNVGCGNVGSSDLVKRMNAGEDPTAVAQSELPQWNKGGGKVLPGLTRRRAAEVALFKTPSSVIAHPAC

SEQ ID NO: 11 - protein, 233 aa, artificial sequence
  synthesized sequence from environmental sample
MDKNIKEIQTLLIQGGFSVGSSGADGLYGNDTRNALIACINKANSGEKKLKLTLEQLNKIFPAGASSGRNAKFIDPLNELFEARQINTVNRIAGFLSQVGVESEEFLYTRELGNAAYFNKYDIQYAPQKAKDLGNTQPGDGAKFKGRGLIQVTGRANYTACGKALGLDLVNRPELLEQPKYAVDSAGWYWGLRNINAACDANDIVAITKKVNGGTMHLDRRTAYYNKAKQVLS

SEQ ID NO: 12 - protein, 209 aa, Sordaria macrospora
TVQGFDISHYQSSVNFAGAYSSGARFVIIKATEGTTYTDPKFSSHYTGATSAGLIRGGYHFAHPDSSTGAAQADYFLAHGGGWSNDGITLPGMIDLESVSGKATCFGLSTSAMVSWIKSFSDRYHTKTGRYPMIYTNYSWWNQCTGNSKTFATTNPLVLARWSSTIGTLPGGWSVHTIWQNADTYTYGGDSDVFNGSLDRLKALAKGSG

SEQ ID NO: 13 - protein, 208 aa, Trichoderma reesei
TVPGFDISHYQATVDFAKAYADGARFVIIKATEGTTYTDPSFSDHYTKATNAGFIRGGYHFAQPASSSGAAQANYFLKHGGGWSADGITLPGMLDLEYAPSGDSCYGLSASAMVSWINDFVNTYHAATTQYPLIYTSTSWWQLCTGNNGSFGSKSPLVIARYASSVGALPNGWSVYTIWQNSDASPWGGDNDIFNGNLAQLQKIARGS

SEQ ID NO: 14 - protein, 196 aa, Agaricomycetes polyporales
APLGKRGSIPGIDVSHYQGSINWSTVKANGVQFVYIKATEGTTYQDPQFSNNYVGATNVGIYRGGYHFARPNLSSGAAQANYFIAHGGGWTSDGRTLPGALDIEYNDDGAECYGLSAASMVSWIKDFSNTYHSKLGVAAFASTNPLWVARYSSSVGTLPAGWSYETFWQHADSGSNPGDQDIFNGDAAGLKRLALG

SEQ ID NO: 15 - protein, 206 aa, Chaetosartorya cremea
TVQGFDISHYQTNVNFAAAYNSGARFVMIKATESTTYIDPSFNSHYTSATSAGFIRGGYHFAVPSDSSGATQANYFLAHGGGWSGDGITLPGMLDIEYNPYGATCYGLSASQMVSWIADFVNTYKSKTGRPPMIYTTADWWNTCTGNSNSFTECPLVLARYSSSVGTIPGGWPYQSFWQNSDSYAYGGDSDIWNGSLDNLKKFASG

SEQ ID NO: 16 - protein, 218 aa, Chaetosartorya cremea
LPSQPEAGATTVQGFDISNHQKSANFEAAKKDGAQFVIIKATEGTTFKDPVFNSHYTGATKAGLIRGGYHFARPDTSTGSAQAKYFLKNGGGWSNDNRTLPGMLDIEYNPYGATCYGLSHSQMVAWIHDFVDEYHHATSRWPMIYTTADWWNRCTGNAKGFGDKCPLVLAAYRSTPPTTIPGDWRTWTIWQNSDKYEHGGDSDKFNGPMKQLRKLASG

SEQ ID NO: 17 - protein, 207 aa, Talaromyces bacillisporus
AVQGFDISSYQGSIDWSGAYSSGARFVIIKATEGTDYIDSGFNSHYTGATNAGFIRGGYHFAHPDSSSGADQAKYFLAHGGGWSNDGITLPGMLDIEYNPSGNECYGLSASAMVSWITDFVNTYHSATGRYPMIYSTDDWWSTCTGNSDAFSSNCPLVLARYGSSPGTIPGGWPYQTIWQNADSYTYGGDSDVFNGSLDNLKKLASG

SEQ ID NO: 18 - protein, 207 aa, Myceliophthora thermophila
AVQGFDISHYQPSVDFAAAYKSGARFVIIKATEGTSYIDPKFSSHYTGATKAGFIRGAYHFAHPGQSSGEAQADYFLAHGGGWTSDGITLPGMLDLEAYNAGQCWGLSTSAMVAWIKAFSDRYHSRTGVYPLLYTNPSWWKACTGNSNAFVNTNPLVLARYASSPGEIPGGWPYQTIWQNSDSYAYGGDSDIFNGDLDGLKRLAKGP

SEQ ID NO: 19 - protein, 219 aa, Tilletiopsis  washingtonensis
APAPEKRASGVQGCDISNYQPNFSYASAADAGAKFVIIKATEGTSYTSPSFSRQYSGATDAGFLRGAYHFAHPDSSSGAAQANFFLANGGGWSGDGRTLPGMVDLEYNPAKNSNSCYGLSQSAMVSWIRSFTSTYYSKTGRYPMIYTTNDWWRTCTGNSDAFSSTSPLVLARYSSSGPGTIPGGWPYQTIWQNSDSFAAGGDSDIFNGSLDGLKRLASG

SEQ ID NO: 20 - protein, 208 aa, Neurospora  tetrasperma
TVQGFDISHYQGSVNFARAYSSGARFVIIKATEGTNYIDPKFSSHYTGATSAGLIRGGYHFAHPDSSSGAAQADYFLAHGGGWSKDGITLPGMIDLESVSGKATCYGLSTSAMVSWIKSFSDRYHSKTGRYPMIYTNYSWWSKCTGNSKSFATTNPLVLARWASSVGTIPGGWSYQTIWQNADTYTYGGDSDIFNGSLDRLKALAKGS

SEQ ID NO: 21 - protein, 215 aa, Trametes  ljubarskyi
SPILEKRANPKGIDISHFQGTVNFNTVKANGISFVYIKATEGTTFTDPDFSSHYTGATNAGLIRGAYHFAHPDVSSGATQAKFFLAHGGGWSSDGITLPGALDIEYNPSGAECYGLSASAMVSWIKDFSNTYHSSTGVYPFIYTTTDWWKTCTGNSAAFASTNPLWIARYASSVGTLPAGWSYHTFWQYADSGPNPGDQDEFNGSMQGLKNLALG

SEQ ID NO: 22 - protein, 234 aa, Penicillium janthinellum
YPVDADDLHCRSGPGTNYGIVKSYKRGTELTITCQAAGTNVNGDELWDKTSDGCYVTDYYVKTGTSGYVTKHCDSGSTGGGGGSSGHGANEATLKLIGQLEGWRPNFYYINGHKTIGYGHDCVEKGCSGINPPLTQQQGLDLLKKDIVGFEDCVCNLPNAKELNANEYGALVSFAYNSGCGGVSRYWHSAMEQKNFKGICEALPHTNTLGGELNNRRKQEGDFCSTPTSEKAGC

SEQ ID NO: 23 - protein, 161 aa, Oidiodendron maius
APSTAIEARNASPINAGAISLIESLEGFRADFYYINGHETIGYGHDCVESGGCGSLHPPISQAEGTALFKKDIAEYESCVCAMANAKDLNANQYGALVSFAYNSGCGGVQSWWHGAMAKKNFKGICEALPTTNTLGGELSSRRKKEGAFCSKATTAKSGCA

SEQ ID NO: 24 - protein, 202 aa, Coniochaeta ligniaria
FDISHYQSSVNYAGAYAAGARFVIIKATEGTTYTDPSFSTHYTGATNAGLIRGGYHFAHPGETTGAAQADYFITHGGGWSGDGITLPGMLDLESEGGATCWGLSTSAMVAWIKAFSDRYHSQTGRYPMLYTNPSWWTSCTGNSNAFVSTNPLVLARYASAPGTIPGGWPYQTIWQNSDSYAYGGDSDIFNGSLDNLRKLATG

SEQ ID NO: 25 - protein, 216 aa, Trinosporium guianense
APALEERASYVQGFDISHYQGTVNFKSAYSSGARFVIIKATEGTSVVDAGFSSHYSGATSAGLIRGGYHFAHPDESSGATQANFFLAHGGGWSNDGITLPGMLDIEYNPSGATCYGLSQSSMVSWIKDFADTYHSKTSRYPLIYTTNDWWTTCTGDSTAFYTTSPLVLARYGSSPGTIPGGWPYETIWQNADSYTYGGDSDKFNGAESSLKKLATG